(19) United States
(12) Patent Application Publication
(10) Pub. No.: US 2016/0130660 A1
(43) Pub. Date: May 12, 2016

(54) COMPOSITIONS AND METHODS FOR MULTIMODAL ANALYSIS OF CMET NUCLEIC ACIDS

(71) Applicant: QIAGEN MANSFIELD, INC., Mansfield, MA (US)

(72) Inventors: Jork NOLLING, Hopedale, MA (US); Kiran Madanahally DIVAKAR, Shrewsbury, MA (US)

(73) Assignee: QIAGEN MANSFIELD, INC., Mansfield, MA (US)

(21) Appl. No.: 14/897,323

(22) PCT Filed: Aug. 7, 2014
(86) PCT No.: PCT/US14/50076

§ 371 Date: Dec. 10, 2015

Related U.S. Application Data
(60) Provisional application No. 61/865,755, filed on Aug. 14, 2013.

(51) Int. Cl. C12Q 1/68

(52) U.S. Cl. CPC C12Q 1/6886; C12Q 2600/156

(57) ABSTRACT

Described herein are methods and assays relating to the detection of cMET alterations (e.g. variations in copy number and expression level, and/or the presence of mutations, including point mutations). Existing methods are limited in their clinical usefulness by, e.g., limited sensitivity, inter-lab discordance, or inability to provide the necessary multiplex ability. The methods and assays provided herein permit multimodal, multiplex assaying for faster, more cost-effective testing and screening of patients, permitting improved healthcare.

### CROSS-REFERENCE TO RELATED APPLICATIONS

This application claims benefit under 35 U.S.C. §119(e) of U.S. Provisional Application No. 61/865,755 filed Aug. 14, 2013, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 31, 2014, is named 046264-077471-PCT_SEtxt and is 97,800 bytes in size.

### TECHNICAL FIELD

The technology described herein relates to assays and methods permitting the detection of cMET alterations (e.g. variations in copy number and expression level, and/or the presence of mutations, including point mutations).

### BACKGROUND

The development of personalized medicine has led to the identification of genes which, when perturbed or altered, can contribute to disease. However, disease-linked genes can be altered in a number of ways, e.g. the expression level of the gene can be altered, the sequence encoding the gene can be altered, and/or the number of genomic copies of the gene (copy number variation; “CNV”) can be altered in a subject who has or is at risk of developing a given disease as compared to a wild-type or healthy subject.

For example, cMET is implicated in cancer and any given cancer cell can demonstrate one or more of these alterations of cMET. Activation of the cMET expression product HGFR (hepatocyte growth factor receptor) contributes to cellular proliferation, cell survival, invasion, cell motility, metastasis, and angiogenesis. Activation of HGFR can be caused by overexpression due to growth factor concentration imbalance, gene amplification, and/or mutations. These alterations of cMET have been found in solid tumors (e.g. renal cancer, gastric cancer, and hepatocellular cancer tumors), adenocarcinoma, and squamous, large cell, and small cell carcinomas.

Detecting each of these types of alterations is typically done using alternative approaches, each of which demonstrates weakness that limit the clinical usefulness. For instance, expression levels are often detected by immunohistochemistry, which can suffer from low antibody sensitivity, resulting in positive samples exhibiting what appear to be weak expression levels. CNV and gene expression levels can be detected by FISH, but these assays can exhibit inter-lab discordance of 20% or more. Mutation and gene expression assays can be conducted by RT-PCR, but existing technologies offer less multiplex ability than is necessary for comprehensive clinical diagnostics. The development of a multimodal, multiplex assay can permit faster, more cost-effective testing and screening of patients, permitting improved healthcare.

### SUMMARY

The technology described herein is directed to methods and assays for detecting alterations of cMET, e.g. alterations in sequence (mutations), expression level, and/or gene copy number. The inventors have developed assays and discovered methods for reliably determining cMET copy number and cMET expression levels in a single multiplexed reaction mixture, and determining cMET copy number, cMET expression levels, and the presence or absence of cMET mutations in a single multiplexed assay comprising as few as two individual reactions.

In one aspect, described herein is an assay for detecting cMET alterations, the assay comprising contacting a portion of a nucleic acid sample with two sets of primers wherein the first set of primers detects alterations in cMET gene copy number variation and the second set of primers detects changes in cMET gene expression level, wherein the first set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation, wherein the second set of primers comprises subsets of primer pairs that amplify mRNA-specific sequences of cMET and mRNA-specific sequences of at least two reference genes, performing a PCR amplification regimen comprising cycles of strand separation, primer annealing, and primer extension on a reaction mixture comprising the portion of the sample and the two sets of primers, detecting the level of the amplicon for each primer pair, normalizing the level of cMET amplicons to the reference gene amplicons. and comparing the normalized level of cMET amplicons to a reference level; wherein a higher level of a gDNA-specific cMET amplicon as compared to the reference level indicates the presence of a gene amplification alteration of cMET in the sample, and an altered level of a mRNA-specific cMET amplicon as compared to the reference level indicates the presence of a gene expression level alteration of cMET in the sample.

In some embodiments, the first set of primers further comprises a subset of primer pairs that amplify at least one gDNA-specific sequence of EGFR and the assay further comprises comparing the normalized level of EGFR amplicons to a reference level; wherein a higher level of a gDNA-specific EGFR amplicon as compared to the reference level indicates the presence of a gene amplification alteration of EGFR in the sample. In some embodiments, the reference gene of the first primer set which is located on chromosome 7 is KDELR-2 and the assay further comprises comparing the normalized level of KDELR-2 amplicons to a reference level; wherein a higher level of a gDNA-specific KDELR-2 amplicon as compared to the reference level indicates the presence of a gene amplification alteration of KDELR-2 in the sample. In some embodiments, the presence of a gene amplification alteration of cMET, EGFR and KDELR-2 indicates the presence of chromosome 7 amplification.

In some embodiments, the reference gene of the first primer set which is not located on chromosome 7 is SOD1 or SPG21. In some embodiments, the first primer set comprises subsets of primer pairs that amplify at least one gDNA-specific sequence of each of SOD1 and SPG21.

In some embodiments, a primer set comprises primer pair subsets that amplify at least one amplicon of each gene. In some embodiments, a primer set comprises primer pair subsets that amplify at least two amplicons of each gene. In some embodiments, a primer set comprises primer pair subsets that amplify at least three amplicons of each gene.

In some embodiments, the primer sets comprise primer pair subsets that amplify at least two gDNA-specific amplicons of each of cMET, EGFR, and KDELR-2 and at least two mRNA-specific amplicons of each of cMET, SOD1 and SGP21. In some embodiments, the primer sets comprise primer pair subsets that amplify at least three gDNA-specific amplicons of each of cMET, EGFR, and KDELR-2 and at least three mRNA-specific amplicons of each of cMET, SOD1 and SGP21.

In some embodiments, the assay can further comprise contacting a second portion of the sample with a third set of primer pairs wherein the third set of primers comprises subsets of primer pairs that amplify cMET sequences comprising sequence variations, performing a PCR amplification regimen comprising cycles of strand separation, primer annealing, and primer extension on a reaction mixture comprising the second portion of the sample and the third set of primers, detecting the level of the amplicon for each primer pair, wherein the presence of an amplicon indicates the presence of the sequence variation for which that primer pair is specific. In some embodiments, the one or more sequence variations of cMET are SNPs. In some embodiments, the cMET SNP is selected from the group consisting of S1058P; V1101I; H1112Y; H1124D; G1137V; M1149T; V1206L; L1213V; K1262R; M1268T; V12381; Y1248C; and D1246N. In some embodiments, S1058P; V1101I; H1112Y; H1124D; G1137V; M1149T; V1206L; L1213V; K1262R; M1268T; V12381; Y1248C; and D1246N are detected.

In some embodiments, the same PCR thermocycling regimens are used for both reactions. In some embodiments, the nucleic acid sample is prepared from a FFPE tumor sample. In some embodiments, the sample comprises tumor cells from a subject diagnosed with a condition selected from the group consisting of gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.

In some embodiments, one or more primers are dual domain primers. In some embodiments, the amplified products from two or more primer pairs of a primer subset can be distinguished. In some embodiments, the amplified products from two or more primer pairs of a primer subset are distinguished by being of distinct sizes. In some embodiments, the amplified products from two or more primer pairs of a primer subset are distinguished by being labeled with different detectable labels. In some embodiments, the amplified products from the first set of primers and the second set of primers are distinguished by being labeled with different detectable labels.

In some embodiments, one or more primers are selected from the group consisting of SEQ ID NOs: 1-83. In some embodiments, one or more primers comprise a sequence of any of SEQ ID NOs: 89-124. In some embodiments, the primers are present in the reaction mixture at about the concentrations of Table 2.

In one aspect, described herein is a method of detecting cMET alterations, the method comprising contacting a portion of a nucleic acid sample with a set of primers which detect alterations in cMET gene copy number variation, wherein the set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation, performing a PCR amplification regimen comprising cycles of strand separation, primer annealing, and primer extension on a reaction mixture comprising the portion of the sample and the set of primers, detecting the level of the amplicon for each primer pair, normalizing the level of cMET amplicons to the reference gene amplicons, and comparing the normalized level of cMET amplicons to a reference level, wherein a higher level of a gDNA-specific cMET amplicon as compared to the reference level indicates the presence of a gene amplification alteration of cMET in the sample.

In some embodiments, the set of primers further comprises a subset of primer pairs that amplify at least one gDNA-specific sequence of EGFR, and the assay further comprises comparing the normalized level of EGFR amplicons to a reference level, wherein a higher level of a gDNA-specific EGFR amplicon as compared to the reference level indicates the presence of a gene amplification alteration of EGFR in the sample. In some embodiments, the reference gene of the primer set which is located on chromosome 7 is KDELR-2; and the method further comprises comparing the normalized level of KDELR-2 amplicons to a reference level; wherein a higher level of a gDNA-specific KDELR-2 amplicon as compared to the reference level indicates the presence of a gene amplification alteration of KDELR-2 in the sample. In some embodiments, the presence of a gene amplification alteration of cMET, EGFR and KDELR-2 indicates the presence of chromosome 7 amplification. In some embodiments, the reference gene of the primer set which is not located on chromosome 7 is SOD1 or SPG21. In some embodiments, the primer set comprises subsets of primer pairs that amplify at least one gDNA-specific sequence of SOD1 and SPG21.

In some embodiments, the method can further comprise contacting the portion of a nucleic acid sample with a second set of primers, wherein the second set of primers detects changes in cMET gene expression level, wherein the second set of primers comprises subsets of primer pairs that amplify mRNA-specific sequences of cMET and at least mRNA specific sequences of at least two reference genes, and wherein an altered level of a mRNA-specific cMET amplicon as compared to the reference level indicates the presence of a gene expression level alteration of cMET in the sample.

In some embodiments, a primer set comprises primer pair subsets that amplify at least one amplicon of each gene. In some embodiments, a primer set comprises primer pair subsets that amplify at least two amplicons of each gene. In some embodiments, a primer set comprises primer pair subsets that amplify at least three amplicons of each gene.

In some embodiments, the primer sets comprise primer pair subsets that amplify at least two gDNA-specific amplicons of each of cMET, EGFR, and KDELR-2 and at least two mRNA-specific amplicons of each of cMET, SOD1 and SGP21. In some embodiments, the primer sets comprise primer pair subsets that amplify at least three gDNA-specific amplicons of each of cMET, EGFR, and KDELR-2 and at least three mRNA-specific amplicons of each of cMET, SOD1 and SGP21.

In some embodiments, the assay can further comprise contacting a second portion of the sample with a third set of primer pairs wherein the third set of primers comprises subsets of primer pairs that amplify cMET sequences comprising sequence variations, performing a PCR amplification regimen comprising cycles of strand separation, primer annealing, and primer extension on a reaction mixture comprising the second portion of the sample and the third set of primers, detecting the level of the amplicon for each primer pair, wherein the presence of an amplicon indicates the presence of the sequence variation for which that primer pair is specific. In some embodiments, the one or more sequence variations of cMET are SNPs. In some embodiments, the cMET SNP is selected from the group consisting of S1058P; V1101I; H1112Y; H1124D; G1137V; M1149T; V1206L; L1213V; K1262R; M1268T; V12381; Y1248C; and D1246N. In some embodiments, S1058P; V1101I; H1112Y; H1124D; G1137V; M1149T; V1206L; L1213V; K1262R; M1268T; V12381; Y1248C; and D1246N are detected.

In some embodiments, the same PCR thermocycling regimens are used for both reactions. In some embodiments, the nucleic acid sample is prepared from a FFPE tumor sample. In some embodiments, the sample comprises tumor cells from a subject diagnosed with a condition selected from the group consisting of gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.

In some embodiments, one or more primers are dual domain primers. In some embodiments, the amplified products from two or more primer pairs of a primer subset can be distinguished. In some embodiments, the amplified products from two or more primer pairs of a primer subset are distinguished by being of distinct sizes. In some embodiments, the amplified products from two or more primer pairs of a primer subset are distinguished by being labeled with different detectable labels. In some embodiments, the amplified products from the first set of primers and the second set of primers are distinguished by being labeled with different detectable labels.

In some embodiments, one or more primers are selected from the group consisting of SEQ ID NOs: 1-83. In some embodiments, one or more primers comprise a sequence of any of SEQ ID NOs: 89-124. In some embodiments, the primers are present in the reaction mixture at about the concentrations of Table 2.

### BRIEF DESCRIPTION OF THE DRAWINGS

FIG. 1 depicts a schematic of an exemplary embodiment of primer targets as described herein.

FIGS. 2 and 3 demonstrate Single Tube CNV and Gene Expression Analysis of gastric cancer cells and depict detection in the TYE and FAM channels, respectively, of an assay using the primers of Table 1 as specified in Table 2.

FIGS. 4 and 5 demonstrate Single Tube CNV and Gene Expression Analysis of lung cancer cells and depict detection in the TYE and FAM channels, respectively, of an assay using the primers of Table 1 as specified in Table 2.

FIG. 6 depicts a graph of the quantified results of an exemplary assay for cMET expression and CNV levels.

FIG. 7 depicts a graph of chromosome 7 polysomy analysis

FIG. 8 depicts a schematic of alternative primer sets for detecting cMET point mutations (e.g. SNPs). FIG. 8 discloses SEQ ID NO: 132.

FIG. 9 depicts the results of a multiplex assay on individual targets uing the shorter amplicon primers of Table 4.

FIG. 10 depicts the results of a multiplex assay on individual targets uing the longer amplicon primers of Table 3.

FIG. 11 depicts the thermocycling parameters used in the assays of Examples 1 and 2.

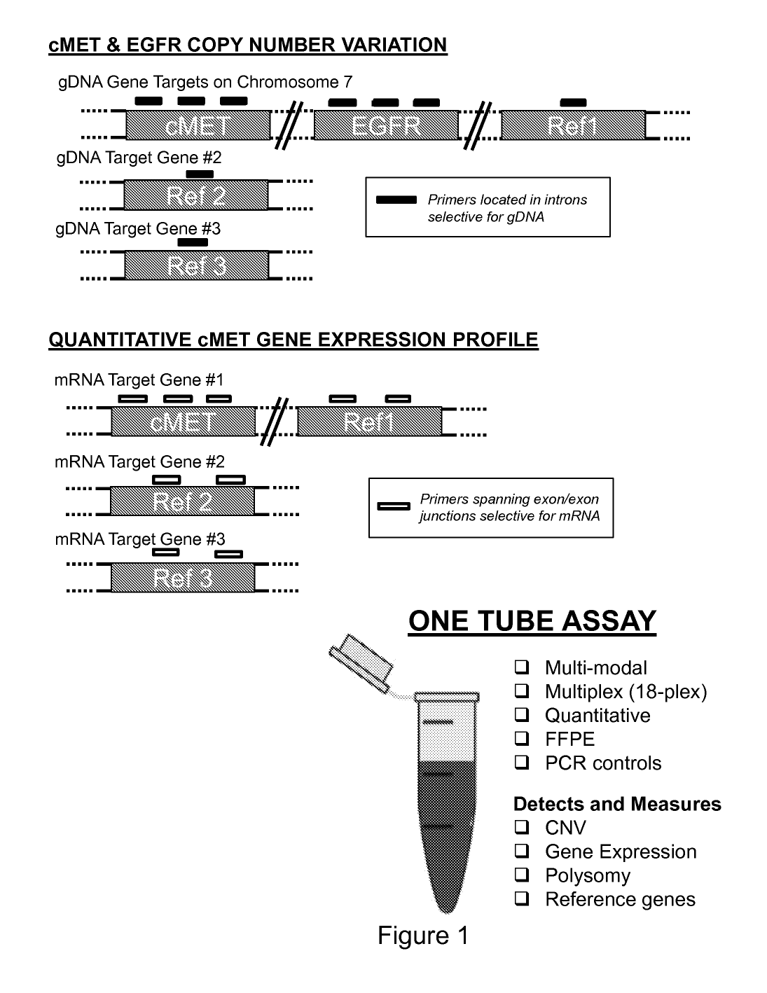

### DETAILED DESCRIPTION

Embodiments of the technology described herein are directed to methods and assays for detecting alterations of cMET, e.g. alterations in sequence (mutations), expression level, and/or gene copy number, and particularly multiplexed and multimodal assays and methods of detecting cMET alterations.

As used herein, the term “HGFR,” “hepatocyte growth factor receptor,” or “cMET” refers to a transmembrane receptor with tyrosine-kinase activity that is activated by binding to hepatocyte growth factor (HGF). The sequences of cMET are well known in the art, eg human cMET (NCBI Gene ID: 4233; SEQ ID NO: 84 (mRNA); SEQ ID NO: 125 (polypeptide)).

As used herein, “alteration”, when used in reference to a gene or gene expression product, refers to a detectable change as compared to the reference (e.g. wild-type) version of that gene or gene expression product, including, but not limited to, changes in gene copy number, changes in expression level, and/or changes in sequence (e.g. sequence variation or mutations).

As used herein “gene copy number” refers to the number of copies of a given gene that occur in the genome. In some embodiments, a single gene and/or a region of a chromosome can be duplicated, e.g. copies of a nucleic acid sequence comprising one or more genes will be found next to each other in the genome or in multiple locations in the genome whereas in a reference genome, one copy of that sequence is present on the relevant chromosome (two copies in a normal diploid genome). In some embodiments, an entire chromosome is duplicated, e.g. polysomy.

As used herein, “expression level” refers to the number of mRNA molecules molecules encoded by a given gene that are present in a cell or sample. Expression levels can be increased or decreased relative to a reference level. Alterations of cMET have been implicated in cancer and detection of such alterations can be of use in diagnosis, prognosis, and/or selection of treatment.

In some embodiments, the assays and/or methods described herein for detecting cMET alterations can comprise contacting a portion of a nucleic acid sample with a set of primers which detect alterations in cMET gene copy number variation, wherein the set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7, to detect cMET gene copy number variation, performing a PCR amplification regimen comprising cycles of strand separation, primer annealing, and primer extension on a reaction mixture comprising the portion of the sample and the set of primers, detecting the level of the amplicon for each primer pair, normalizing the level of cMET amplicons to the reference gene amplicons, thereby determining the relative level of cMET copy number. In some embodiments, the relative level of cMET copy number can be compared to a reference level (e.g. a pre-determined reference level); wherein a higher relative level of one or more gDNA-specific cMET amplicon as compared to the reference level indicates the presence of a gene amplification alteration of cMET in the sample. In some embodiments, the methods and assays can further comprise contacting a portion of a nucleic acid sample with a second set of primers, wherein the second set of primers detects changes in cMET gene expression level, wherein the second set of primers comprises subsets of primer pairs that amplify mRNA-specific sequences of cMET and, optionally, at least mRNA specific sequences of at least two reference genes, and normalizing the level of cMET amplicons to the reference gene amplicons, thereby determining the relative level of cMET expression. In some embodiments, the relative level of cMET expression can be compared to a reference level (e.g. a pre-determined reference level); wherein a higher relative level of one or more mRNA-specific cMET amplicon as compared to the reference level indicates the presence of a gene expression alteration of cMET in the sample.

In some embodiments, the assays and/or methods described herein for detecting cMET alterations comprise contacting a portion of a nucleic acid sample with two sets of primers wherein the first set of primers detects alterations in cMET gene copy number variation and the second set of primers detects changes in cMET gene expression level; wherein the first set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation; wherein the second set of primers comprises subsets of primer pairs that amplify mRNA-specific sequences of cMET and at least mRNA specific sequences of at least two reference genes; performing a PCR amplification regimen comprising cycles of strand separation, primer annealing, and primer extension on a reaction mixture comprising the portion of the sample and the two sets of primers; detecting the level of the amplicon for each primer pair; normalizing the level of cMET amplicons to the reference gene amplicons; and comparing the normalized level of cMET amplicons to a reference level of cMET; wherein a higher level of a gDNA-specific cMET amplicon as compared to the reference level of cMET indicates the presence of a gene amplification alteration of cMET in the sample, and an altered level of a mRNA-specific cMET amplicon as compared to the reference level of cMET indicates the presence of a gene expression level alteration of cMET in the sample.

In some embodiments, the assays described herein occur in a single tube, e.g. the first and second sets of primers are present in a single reaction mixture and/or vessel or container. Thus, in said embodiments, a single amplification regimen will provide data regarding gene copy number and gene expression level.

In some embodiments, the first set of primers further comprises a subset of primer pairs that amplify at least one gDNA-specific sequence of EGFR and the method comprises comparing the normalized level of EGFR amplicons to a reference level; wherein a higher level of a gDNA-specific EGFR amplicon as compared to the reference level indicates the presence of a gene amplification alteration of EGFR in the sample. As used herein, the term “EGFR” or “Epiderm Growth Factor Receptor” refers to a transmembrane receptor that binds to ligands including epidemeral growth factor “EGF” and TGFα. Ligand recognition causes autophosphorylation of EGFR and activates the MAPK, Akt, and/or JNK pathways, leading to cellular proliferation. The sequences of EGFR are well known in the art, eg. human EGFR (NCBI Gene ID: 1956; SEQ ID NO: 85 (mRNA); SEQ ID NO: 126 (polypeptide)).

Alterations of EGFR, e.g. an increase in gene copy number of EGFR have been implicated in cancer and detection of such alterations can be of use in diagnosis, prognosis, and/or selection of treatment. In some embodiments, the gene copy number of cMET and EGFR are detected in the same reaction mixture, e.g. in the same tube, well, or vessel.

In order to reliably detect a level of cMET (and, optionally, EGFR), e.g. a gene copy number level and/or an expression product level, one can normalize the level of cMET in a sample to the copy number or expression level, respectively, of one or more reference genes. In some embodiments, a reference gene can be a gene which is not typically subject to alterations in cancer cells. The normalized level can then be compared to a reference level for the target gene, e.g. the level of the gene in a normal, healthy, and/or reference sample.

The terms “reference level” and “reference sample” are used interchangeably herein and refer to the expression level of copy number signal of a gene in a known sample against which a second sample (i.e. one obtained from a subject) is compared. A reference level is useful for determining the presence and magnitude of an alteration in, e.g. cMET in a biological sample comprising nucleic acids. A reference value serves as a reference level for comparison, such that samples can be normalized to an appropriate standard in order to infer the presence, absence or extent of an alteration in a sample. In some embodiments, a reference level can be a level that was previously determined, e.g. the reference level can be a pre-determined number or ratio and need not be determined in the same physical iteration of an assay as described herein.

A reference level can be obtained, for example, from a known biological sample from a subject that is e.g., substantially free of cancer and/or who does not display any symptoms or risk factors for having cancer. A known sample can also be obtained by pooling samples from a plurality of individuals to produce a reference value or range of values over an averaged population, wherein a reference value represents an average level of, e.g. gene copy number, or expression level among a population of individuals (e.g., a population of individuals not having cancer). Thus, the level of a gene copy number or gene expression in a reference obtained in this manner is representative of an average level in a general population of individuals not having cancer. In some embodiments, the reference value can be the level in an equivalent sample obtained from a healthy adult subject. As used herein, a “healthy adult subject” can be one who does not display any markers, signs, or symptoms of cancer and who is not at risk of having cancer. In some embodiments, the population of healthy adult subjects can include subjects with similar demographic characteristics as the subject, e.g. similar age, similar ethnic background, similar diets, etc.

In the methods and assays described herein, the relative copy number and/or expression level of a target gene (e.g. cMET) can be determined by comparison to a reference gene, as described below herein. A reference gene can be, preferably, one that is not typically altered (either in expression level or copy number) in cells which are affected by the disease of interest relative to healthy cells.

The reference gene can be a gene which is not subject to alteration in diseased cells (e.g. cancer cells, gastric cancer cells, renal cancer cells, cholangioma cells, lung cancer cells, brain cancer cells, cervical cancer cells, colon cancer cells, head and neck cancer cells, hepatoma cancer cells, non-small cell lung cancer cells, melanoma cells, mesothelioma cells, multiple myeloma cells, ovarian cancer cells, sarcoma cells, and/or thyroid cancer cells) as compared to healthy (e.g. non-cancerous) cells.

Where the reference gene is a polysomy reference gene not located on chromosome 7, it is preferable that the polysomy reference gene is located on a chromosome that is not subject to polysomy, or not known to be subject to polysomy in diseased cells (e.g. cancer cells, gastric cancer cells, renal cancer cells, cholangioma cells, lung cancer cells, brain cancer cells, cervical cancer cells, colon cancer cells, head and neck cancer cells, hepatoma cancer cells, non-small cell lung cancer cells, melanoma cells, mesothelioma cells, multiple myeloma cells, ovarian cancer cells, sarcoma cells, and/or thyroid cancer cells) as compared to healthy (e.g. non-cancerous) cells.

When detecting the gene copy number level of a target gene (e.g. cMET), the level of amplicons produced by a primer pair subset specific for a gDNA-specific sequence of a target gene can be compared to each of two polysomy references from the same sample. The first polysomy reference is the level of amplicons produced by a primer pair subset specific for a gDNA-specific sequence of a gene present on the same chromosome as the target gene. The second polysomy reference is the level of amplicons produced by a primer pair subset specific for a gDNA-specific sequence of a gene present on a different chromosome than the target gene and the first polysomy reference gene. If the level detected for the target gene is greater than the level dectected for the first polysomy reference gene, it indicates that extra copies of the target gene, or a portion of the chromosome comprising the target gene but not the same-chromosome reference gene are present in the genome. If the levels detected for the target gene and the first reference gene are greater than the level dectected for the second reference gene, it indicates that extra copies of the chromosome comprising the target gene and the first polysomy reference gene are present in the sample (e.g. polysomy is indicated for the chromosome comprising the target gene).

For example, in some embodiments, the presence of a gene copy number alteration of cMET, but not of any of the polysomy reference genes present on chromosome 7 indicates that cMET has been subject to gene amplification. In some embodiments, the presence of a gene copy number alteration of the polysomy reference gene(s) present on chromosome 7, but not of any of the polysomy reference genes not present on chromosome 7 indicates the presence of polysomy of chromosome 7, e.g. extra copies of the entire chromosome 7 or parts of it are present in the cell(s) from which the nucleic acid sample was obtained. In some embodiments, if gene copy number alterations are detected for both cMET and the polysomy reference gene(s) present on chromosome 7, both polysomy and amplification of cMET (or a region comprising cMET) can be indicated for the nucleic acid sample. When the level of gDNA-specific amplicons for a given gene (e.g. cMET, EGFR, and/or KDELR-2) is compared to the polysomy reference gene and/or polysomy reference level, the magnitude of the level of difference (fold difference) between the gene copy number level of a gene on chromosome 7 and the reference can be determined.

A similary approach can be used to detect the presence and/or magnitude of a gene expression alteration. When detecting the expression level of a target gene (e.g. cMET), the level of amplicons produced by a primer pair subset specific for an mRNA-specific sequence of a target gene can be normalized to the expression level of at least one reference gene from the same sample. Once normalized to the expression level of the reference gene(s), the expression level of the target gene can be compared to a reference expression level for the target gene, e.g. the expression level of the target gene in a healthy, non-cancerous cell and/or tissue sample. In some embodiments, the reference level can be pre-determined.

In some embodiments, the reference gene for determining the gene expression level of cMET can be SOD1 and/or SPG21. In some embodiments, an assay or method described herein can comprise determining the level of SOD1 and/or SPG21 mRNA in a nucleic acid sample, e.g. contacting the sample with primer sets specific for SOD1 and/or SPG21 sequences, performing PCR amplification of the SOD1 and/or SPG21 target(s), and detecting the level of resulting amplicons.

As used herein, “superoxide disumutase 1” or “SOD1” refers to a dismutase that destroys superoxide radicals. The sequences of SOD1 are well known in the art, e.g. human SOD1 (NCBI Gene ID: 6647; SEQ ID NO: 87 (mRNA); SEQ ID NO: 127 (polypeptide)).

As used herein, “spastic paraplegia 21” or “SPG21” refers to a negative regulator of CD4 that directly binds to CD4. The sequences of SPG21 are well known in the art, eg. human SPG21 (NCBI Gene ID: 51324; SEQ ID NO: 88 (mRNA); SEQ ID NO: 128 (polypeptide)).

In some embodiments, the reference gene(s) for determining the gene copy number level of cMET can include at least one reference gene on chromosome 7 and at least one reference gene not on chromosome 7. In some embodiments, the reference genes for determining the gene copy number level of cMET can include one reference gene on chromosome 7 and one reference gene not on chromosome 7. In some embodiments, the reference genes for determining the gene copy number level of cMET can include two reference genes on chromosome 7 and two reference genes not on chromosome 7. In some embodiments, the reference gene(s) present on chromosome 7 can be EGFR and/or KDELR-2. In some embodiments, the reference genes(s) not present on chromsomone 7 can be SOD1 and/or SPG21.

As used herein, “ER lumen protein retaining receptor 2” or “KDELR-2” refers to a receptor that binds to proteins in the cis-Golgi or pre-Golgi compartment via a tetrapeptide signal (KDEL (SEQ ID NO: 130)) and cause the bound proteins to be moved to the ER lumen. The sequences of KDELR-2 are well known in the art, eg. human KDELR-2 (NCBI Gene ID: 11014; SEQ ID NO: 86 (mRNA); SEQ ID NO: 129 (polypeptide)).

In some embodiments, the reference gene(s) not located on chromosome 7 can be SOD1 and/or SPG21. In some embodiments, the first set of primers comprises at least one set of primers specific for a gDNA-specific sequence of SOD1 or SPG21. In some embodiments, the first set of primers comprises at least one set of primers specific for a gDNA-specific sequence of each of SOD1 and SPG21.

In some embodiments, wherein KDELR-2 is a reference gene on chromosome 7, and the normalized level of KDELR-2 amplicon(s) is compared to a reference level, a higher level of a gDNA-specific KDELR-2 amplicon(s) as compared to the reference level indicates the presence of a gene copy number alteration of KDELR-2 in the sample and/or the presence of polysomy of chromosome 7.

In some embodiments, the accuracy and reliability of the assays and methods described herein can be improved by detecting multiple sequences from within each of the target genes, e.g. a set of primers can contain multiple subsets of primers which are specific for separate sequences of the same gene so that after PCR amplification, multiple amplicons derived from each target gene are present. This is expected to improve assay accuracy. In some embodiments, the level of a given target gene, e.g. the gene copy number level or the gene expression level can be determined by averaging and/or taking the geometric mean of the level of multiple amplicons, e.g. before normalization and comparison to the reference level.

In some embodiments, a primer set can comprise primer pair subsets that amplify at least one amplicon of each gene. In some embodiments, a primer set can comprise primer pair subsets that amplify at least two amplicons of each gene. In some embodiments, a primer set can comprise primer pair subsets that amplify at least three amplicons of each gene.

In some embodiments, the primer sets can comprise primer pair subsets that amplify at least two gDNA-specific amplicons of each of cMET, EGFR, and KDELR-2 and at least two mRNA-specific amplicons of each of cMET, SOD1 and SGP21. In some embodiments, the primer sets can comprise primer pair subsets that amplify at least three gDNA-specific amplicons of each of cMET, EGFR, and KDELR-2 and at least three mRNA-specific amplicons of each of cMET, SOD1 and SGP21.

In some embodiments, the assays and methods described herein can further comprise detecting the presence of sequence variations in cMET. As used herein, “sequence variations” can refer to substitutions, insertions, deletions, duplications, or rearrangements.

A sequence variation, including, e.g. a point mutation, e.g. a single nucleotide polymorphism (SNP), can be phenotypically neutral or can have an associated variant phenotype that distinguishes it from that exhibited by the predominant sequence at that locus. As used herein, “neutral polymorphism” refers to a polymorphism in which the sequence variation does not alter gene function, and “mutation” or “functional polymorphism” refers to a sequence variation which does alter gene function, and which thus has an associated phenotype. Sequence variations of a locus occurring in a population are referred to as alleles. When referring to the genotype of an individual with regard to a specific locus at which two or more alleles occur within a population, the “predominant allele” is that which occurs most frequently in the population in question (i.e., when there are two alleles, the allele that occurs in greater than 50% of the population is the predominant allele; when there are more than two alleles, the “predominant allele” is that which occurs in the subject population at the highest frequency, e.g., at least 5% higher frequency, relative to the other alleles at that site). The term “variant allele” is used to refer to the allele or alleles occurring less frequently than the predominant allele in that population (e.g., when there are two alleles, the variant allele is that which occurs in less than 50% of the subject population; when there are more than two alleles, the variant alleles are all of those that occur less frequently, e.g., at least 5% less frequently, than the predominant allele). Sequence variations can be present in (and therefore, detected in) the gDNA and/or mRNA of a gene.

In some embodiments, the sequence variant can be a point mutation. As used herein, a “point mutation” refers to the identity of the nucleotide present at a site of a mutation in the mutant copy of a genomic locus (including insertions and deletions), i.e. it refers to an alteration in the sequence of a nucleotide at a single base position from the wild type sequence. A SNP (single nucleotide polymorphism) is one type of point mutation that occurs at the same genomic locus between different individuals in a population. Point mutations may be somatic in that they occur between different cells in the same individual.

In some embodiments, the sequence variation can be a single nucleotide polymorphism (SNP). As used herein, a “single nucleotide polymorphism” or “SNP” refers to nucleic acid sequence variation at a single nucleotide residue, including a single nucleotide deletion, insertion, or base change or substitution. SNPs can be allelic. Some SNPs have defined phenotypes, e.g. disease phenotypes, while others have no known associated phenotype. SNP detection methods, described herein can be used for the prediction of phenotypic characterisitics, e.g. prediction of responsiveness or sensitivity to drugs. In this regard, SNP genotyping as described herein and known in the art is not necessarily diagnostic of disease or susceptibility to disease.

As noted, in some embodiments, an alteration comprises a SNP. At least four alleles of a SNP locus are possible, although SNPs that vary only between two nucleotides at the target site are not uncommon. In some embodiments, the methods and compositions described herein relate to a subset of primer pairs that can detect a single allele of a SNP locus. In some embodiments, the methods and compositions described herein relate to a set of primers that can detect two alleles of a SNP locus (i.e. the methods and compositons can relate to an assay that permits the affirmative detection of two SNP alleles, or “biphasic” genotyping of that SNP). In some embodiments, the methods and compositions described herein relate to a set of primers that can detect three alleles of a SNP locus (i.e. the methods and compositons can relate to an assay that permits the affirmative detection of three SNP alleles, or “triphasic” genotyping of that SNP). In some embodiments, the methods and compositions described herein relate to an assay that permits affirmative detection of four alleles of a SNP locus (i.e. the methods and compositons can relate to a multiplex detection of four SNP alleles, or “quaduphasic” genotyping of that SNP). In some embodiments, the predominant and/or wild-type allele of a SNP is detected. In some embodiments, the predominant and/or wild-type allele of a SNP is not detected. By “affirmatively detected” is meant that the assay permits the amplification of that specific allele. An alternative to affirmative detection can be used, for example, when there are only two possibilities known to exist at the SNP site. In this instance, the assay can be designed such that one of the two variants is amplified, and the other is not; the assay can affirmatively detect that amplified variant and passively detect the other, i.e. the lack of a product means the other allele or variant is present.

In some embodiments, an assay or method described herein can further comprise contacting a second portion of the sample with a third set of primer pairs wherein the third set of primers comprises subsets of primer pairs that amplify cMET sequences comprising sequence variations; performing a PCR amplification regimen comprising cycles of strand separation, primer annealing, and primer extension on a reaction mixture comprising the second portion of the sample and the third set of primers; and detecting the level of the amplicon for each primer pair, wherein the presence of an amplicon indicates the presence of the sequence variation for which that primer pair is specific. In some embodiments, the reaction comprising the first portion of the sample and the first (and optionally, second) primer sets and the reaction comprising the second portion of the sample and the third primer set can be performed using the same thermocycling conditions, e.g. the two reactions can be performed simultaneously in separate wells of the same multi-well plate or can be performed simultaneously in separate tubes in the same machine or parallel machines using the same set of thermocycling conditions.

In some embodiments, the cMET sequence variation(s) can be SNPs. In some embodiments, a cMET SNP can be a SNP resulting in the following amino acid residue changes: S1058P; V1101I; H1112Y; H1124D; G1137V; M1149T; V1206L; L1213V; K1262R; M1268T; V12381; Y1248C; and/or D1246N. In some embodiments, an assay or method described herein comprises a third primer set that can specifically amplify one or more of the SNPs resulting in the following amino acid residue changes: S1058P; V1101I; H1112Y; H1124D; G1137V; M1149T; V1206L; L1213V; K1262R; M1268T; V12381; Y1248C; and/or D1246N.

In various embodiments, the methods and compositions described herein relate to performing a PCR amplification regimen with at least one set of oligonucleotide primers. As used herein, “primer” refers to a DNA or RNA polynucleotide molecule or an analog thereof capable of sequence-specifically annealing to a polynucleotide template and providing a 3′ end that serves as a substrate for a template-dependent polymerase to produce an extension product which is complementary to the polynucleotide template. The conditions for initiation and extension usually include the presence of at least one, but more preferably all four different deoxyribonucleoside triphosphates and a polymerization-inducing agent such as DNA polymerase or reverse transcriptase, in a suitable buffer (in this context “buffer” includes solvents (generally aqueous) plus necessary cofactors and reagents which affect pH, ionic strength, etc.) and at a suitable temperature. A primer useful in the methods described herein is generally single-stranded, and a primer and its complement can anneal to form a double-stranded polynucleotide. Primers according to the methods and compositions described herein can be less than or equal to 300 nucleotides in length, e.g., less than or equal to 300, or 250, or 200, or 150, or 100, or 90, or 80, or 70, or 60, or 50, or 40, and preferably 30 or fewer, or 20 or fewer, or 15 or fewer, but at least 10 nucleotides in length.

As used herein, the term “set” means a group of nucleic acid samples, primers or other entities. A set will comprise a known number of, and at least two of such entities. A set of primers comprises at least one forward primer and at least one reverse primer specific for a target sequence. A set of primers will comprise at least one primer pair subset, e.g. one primer pair subset, two primer pair subsets, three primer pair subsets, four primer pair subsets, five primer pair subsets, six primer pair subsets, or more primer pair subsets. A set of primers comprises the group of primer pair subsets that detect the same type of alteration, e.g. the primer pair subsets that can detect gene copy number levels, expression levels, or sequence variations. A set of primers can comprise primer pair subsets that detect the same type of alterations in different genes, e.g. a primer set can comprise two primer pair subsets, one of which detects gene copy number levels in cMET and the other of which detects gene copy number levels in KDELR-2.

Thus, as used herein, “a primer pair subset” refers to a group of at least two primers, including a forward primer and a reverse primer, one of which anneals to a first strand of a target nucleic acid sequence and the other of which anneals to a complement of the first strand. In some embodiments, the first primer of a primer pair subset can anneal to a first strand of a target nucleic acid sequence and the second primer of a primer pair subset (e.g., reverse primer), can anneal to the complement of that strand. The orientation of the primers when annealed to the target and/or its complement can be such that nucleic acid synthesis proceeding from primer extension of a one primer of the primer pair subset would produce a nucleic acid sequence that is complementary to at least one region of the second primer of the primer pair subset. The “first strand” of a nucleic acid target and/or sequence can be either strand of a double-stranded nucleic acid comprising the sequence of the target nucleotide and/or target site locus, but once chosen, defines its complement as the second strand. Thus, as used herein, a “forward primer” is a primer which anneals to a first strand of a nucleic acid target, while a “reverse primer” of the same set is a primer which anneals to the complement of the first strand of the nucleic acid target.

As used herein, “specific” when used in the context of a primer specific for a target nucleic acid refers to a level of complementarity between the primer and the target such that there exists an annealing temperature at which the primer will anneal to and mediate amplification of the target nucleic acid and will not anneal to or mediate amplification of non-target sequences present in a sample. In the context of primer pair subsets that amplify sequence variations, at least one of the primers of the subset is specific for the sequence variation, e.g. the primer pair subset will not amplify the wild-type sequence not comprising the sequence variation.

In some embodiments, in order to specifically detect mRNA or cDNA in the presence of gDNA, one or more mRNA-specific primers can be intron-spanning primers. As used herein, a primer pair subset is “mRNA-specific” if it amplifies an amplicon from mRNA and/or cDNA but not from gDNA or if the amplicon amplified from mRNA and/or cDNA is distinguishable in size from the amplicon amplified from gDNA. A mRNA-specific primer pair subset that amplifies an amplicon from mRNA and/or cDNA but not from gDNA can include, e.g. at least one primer that specifically binds to an exon-exon boundary of an mRNA or cDNA, e.g. such that it can specifically bind to an mRNA or cDNA in which the introns have been removed, but not to gDNA in which the introns are present. A mRNA-specific primer pair subset that amplifies an amplicon from mRNA and/or cDNA is distinguishable in size from the amplicon amplified from gDNA can include, e.g. primers that specifically bind to sequences which flank one or more introns, such that the distance between the sequences specifically bound by the primer pair subset is larger in the gDNA than in the mRNA or cDNA lacking the one or more introns. In some embodiments, in order to specifically detect gDNA in the presence of RNA or cDNE, one or more gDNA-specific primers can specifically anneal to the intron of a target nucleic acid sequence. As used herein, a primer pair subset is “gDNA-specific” if it specifically amplifies an amplicon from gDNA but not from mRNA or cDNA. In some embodiments, in order to detect short target polynucleotides (e.g. miRNAs or degraded target polynucleotides) as well as longer target polynucleotides (e.g. mRNA or target site loci in genomic DNA), primers for at least the shorter target polynucleotides can comprise tag sequence that results in an amplified product of larger, discrete size than the target sequence. The tags can be designed such that all amplified products in a reaction will be of distinct sizes.

Methods of making primers are well known in the art, and numerous commercial sources offer oligonucleotide synthesis services suitable for providing primers according to the methods and compositions described herein, e.g. INVITROGEN™ Custom DNA Oligos; Life Technologies; Grand Island, N.Y. or custom DNA Oligos from IDT; Coralville, Iowa).

In some embodiments, one or more primers can be dual domain primers. Dual domain primers are described in detail in PCT/US13/27383, filed Feb. 22, 2013; the contents of which are incorporated by reference herein in its entirety.

Exemplary embodiments of primers are described herein. In some embodiments, one or more primers can be selected from the group consisting of SEQ ID NOs: 1-83. In some embodiments, one or more primers of the first set of primers can be selected from the group consisting of SEQ ID NOs: 10-18 and 28-36. In some embodiments, one or more primers of the second set of primers can be selected from the group consisting of SEQ ID NOs: 1-10, 19-27, and 37-45. Exemplary subsets of primer pairs for the first and second sets of primers are depicted in Table 2. In some embodiments, one or more primers of the third set of primers can be selected from the group consisting of SEQ ID NOs: 46-64. In some embodiments, one or more primers of the third set of primers can be selected from the group consisting of SEQ ID NOs: 64-83. In some embodiments, the primers can be present in the reaction mixture(s) at about the concentrations of Table 2. In some embodiments, one or more primers comprise a sequence of any of SEQ ID NOs: 89-124.

The methods and compositions described herein relate to performing a polymerase chain reaction (PCR) amplification regimen. As used herein, the term “amplification regimen” refers to a process of specifically amplifying, i.e., increasing the abundance of, a nucleic acid sequence of interest, and more particularly, the exponential amplification occurring when the products of a previous polymerase extension serve as templates for the successive rounds of extension. A PCR amplification regimen according to the invention comprises at least two, and preferably at least 5, 10, 15, 20, 25, 30, 35 or more iterative cycles, where each cycle comprises the steps of: 1) strand separation (e.g., thermal denaturation); 2) oligonucleotide primer annealing to template molecules; and 3) nucleic acid polymerase extension of the annealed primers. Conditions and times necessary for each of these steps can be devised by one of ordinary skill in the art. An amplification regimen according to the methods described herein is preferably performed in a thermal cycler, many of which are commercially available.

In some embodiments, the nucleic acid sample can be subjected to reverse transcription prior to the PCR amplification regimen described herein, e.g. when the level of an mRNA is to be determined as described herein. Reverse transcription protocols and reagents are well known in the art and are commercially available. An exemplary embodiment of a reverse transcription regimen is as follows: 5 uL of a nucleic acid sample comprising both RNA and gDNA (e.g. 25 ng of RNA and 2.5 ng of gDNA) are added to a reaction mixture comprising RT buffer, 0.5 mM dNTPs, 5 nM RT primers, and 20 units of SuperScript III™ reverse transcriptase (RNA-dependent DNA polymerase). The reaction is then incubated at 50° C. for 30 minutes, 90° C. for 5 minutes, and 4° C. for 5 minutes. Exemplary embodiments of RT primers suitable for use in the methods and assays are described in the Examples herein, e.g. SEQ ID NOs: 1-9.

PCR requires the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of preferred nucleic acid polymerases are thermostable, i.e., they retain function after being subjected to temperatures sufficient to denature annealed strands of complementary nucleic acids, e.g. 94° C., or sometimes higher. In some embodiments, the polymerase can be delta-exo-Apta Taq Polymerase.

As understood in the art, PCR requires cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. More specifically, strand separation according to the methods described herein is achieved by heating the nucleic acid sample above its Tₘ. Generally, for a sample containing nucleic acid molecules in buffer suitable for a nucleic acid polymerase, heating to 94° C. is sufficient to achieve strand separation. An exemplary buffer contains 50 mM KCl, 10 mM Tric-HCl (pH 8.8@25° C.), 0.5 to 3 mM MgCl₂, and 0.1% BSA.

As also understood in the art, PCR requires annealing primers to template nucleic acids. As used herein, “anneal” refers to permitting two complementary or substantially complementary nucleic acids strands to hybridize, and more particularly, when used in the context of PCR, to hybridize such that a primer extension substrate for a template-dependent polymerase enzyme is formed. Conditions for primer-target nucleic acid annealing vary with the length and sequence of the primer and are based upon the calculated Tₘ for the primer. Generally, an annealing step in an amplification regimen involves reducing the temperature following the strand separation step to a temperature based on the calculated Tₘ for the primer sequence, for a time sufficient to permit such annealing.

Tₘ can be readily predicted by one of skill in the art using any of a number of widely available algorithms (e.g., OLIGO™ (Molecular Biology Insights Inc. Colorado) primer design software and VENTRO NTI™ (Invitrogen, Inc. California) primer design software and programs available on the internet, including Primer3 and Oligo Calculator). For example, Tₘ's can be calculated using the NetPrimer software (Premier Biosoft; Palo Alto, Calif.; and freely available on the world wide web at http://www.premierbiosoft.com/netprimer/netprlaunch/Help/xnetprlaunch.html). The Tₘ of a primer can also be calculated using the following formula, which is used by NetPrimer software and is described in more detail in Frieir et al. PNAS 1986 83:9373-9377 which is incorporated by reference herein in its entirety.

Tₘ=ΔH/(ΔS+R*ln(C/4))+16.6 log([K⁺]/(1+0.7[K⁺]))−273.15

wherein, ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). Generally, the closer the annealing temperature is to the Tₘ, the more specific is the annealing. The time allowed for primer annealing during a PCR amplification regimen depends largely upon the volume of the reaction, with larger volumes requiring longer times, but also depends upon primer and template concentrations, with higher relative concentrations of primer to template requiring less time than lower relative concentrations. Depending upon volume and relative primer/template concentration, primer annealing steps in an amplification regimen can be on the order of 1 second to 5 minutes, but will generally be between 10 seconds and 2 minutes, preferably on the order of 30 seconds to 2 minutes.

As used herein, “substantially anneal” refers to a degree of annealing during a PCR amplification regimen which is sufficient to produce a detectable level of a specifically amplified product.

PCR also relies upon polymerase extension of annealed primers at each cycle. As used herein, the term “polymerase extension” means the template-dependent incorporation of at least one complementary nucleotide, by a nucleic acid polymerase, onto the 3′ end of an annealed primer. Polymerase extension preferably adds more than one nucleotide, preferably up to and including nucleotides corresponding to the full length of the template. Conditions for polymerase extension vary with the identity of the polymerase. The temperature used for polymerase extension is generally based upon the known activity properties of the enzyme. Although, where annealing temperatures are required to be, for example, below the optimal temperatures for the enzyme, it will often be acceptable to use a lower extension temperature. In general, although the enzymes retain at least partial activity below their optimal extension temperatures, polymerase extension by the most commonly used thermostable polymerases (e.g., Taq polymerase and variants thereof) is performed at 65° C. to 75° C., preferably about 68-72° C.

Primer extension is performed under conditions that permit the extension of annealed oligonucleotide primers. As used herein, the term “conditions that permit the extension of an annealed oligonucleotide such that extension products are generated” refers to the set of conditions including, for example temperature, salt and co-factor concentrations, pH, and enzyme concentration under which a nucleic acid polymerase catalyzes primer extension. Such conditions will vary with the identity of the nucleic acid polymerase being used, but the conditions for a large number of useful polymerase enzymes are well known to those skilled in the art. One exemplary set of conditions is 50 mM KCl, 10 mM Tric-HCl (pH 8.8@25° C.), 0.5 to 3 mM MgCl₂, 200 uM each dNTP, and 0.1% BSA at 72° C., under which Taq polymerase catalyzes primer extension.

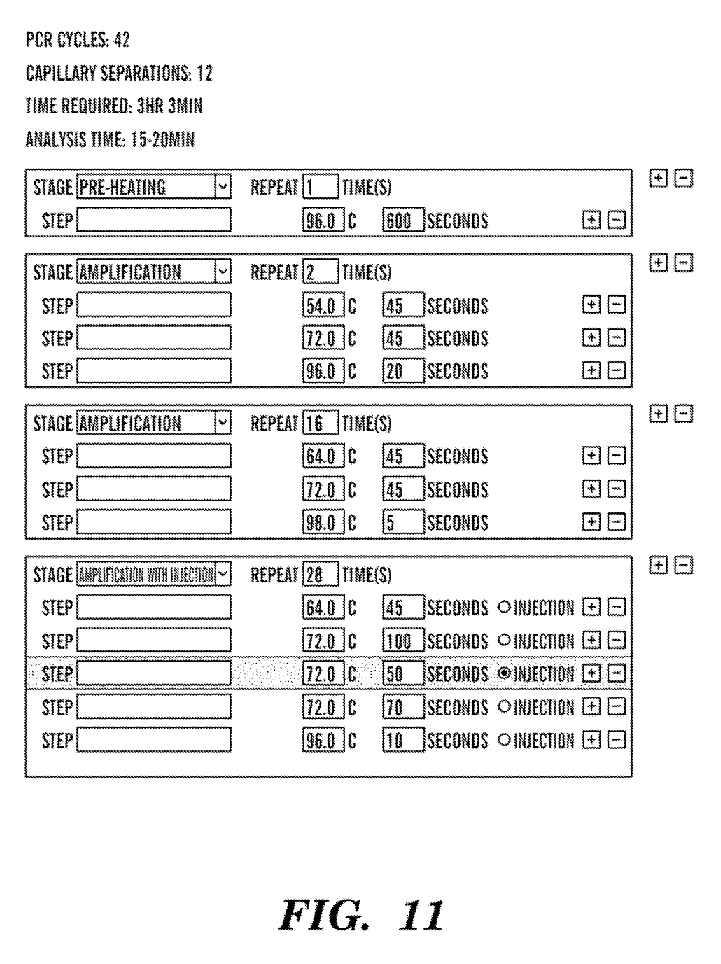

In some embodiments, the thermocycling conditions can be in accordance with the protocol depicted in FIG. 11.

In some embodiments, a buffer for use in the methods and assays described herein can comprise Tris buffer, trehalose, potassium acetate, glycerol, betaine, magnesium chloride, potassium chloride, ammonium sulphate, DMSO, DTT, BSA, dNTPs, Tween-20 and polymerase. In some embodiments, a buffer for use in the methods and assays described herein can comprise 10-400 mM Tris buffer (pH 7.5 to 9.5), 2-20% trehalose, 10-300 mM potassium acetate, 1-7.5% glycerol, 100 mM to 2M betaine, 2.5-12.5 mM magnesium chloride, 1-10 mM potassium chloride, 1-10 mM ammonium sulphate, 0.1-2% DMSO, 1-10 mM DTT, 10-1,000 ug/mL BSA, 50-400 mM dNTP, 0-1% Tween-20 and 1-10 enzyme units of polymerase.

As used herein, “amplified product” or “amplicon” refers to polynucleotides resulting from a PCR reaction that are copies of a portion of a particular target nucleic acid sequence and/or its complementary sequence, which correspond in nucleotide sequence to the template nucleic acid sequence and/or its complementary sequence. An amplified product, as described herein will generally be double-stranded DNA, although reference can be made to individual strands thereof.

The methods described herein use PCR to quantitate or eavlaute gene copy number and variations thereof, as well as for quantitation or evaluation of gene expression and/or gene mutation. For any of the methods described ehrein, quantiation can be achieved by withdrawing samples from the PCR reaction at plural cycles and separating and detecting the amounts of the amplicons in the sample withdrawn. The amplification profile for each amplicon measured in this manner permits the quantitation of initial template. See, e.g., U.S. Pat. No. 8,321,140 and U.S. Patent Application No. 2013/0053274; which are incorporated by reference herein in their entireties.

In some embodiments, the methods and compositions described herein relate to multiplex PCR. As used herein, “multiplex PCR” refers to a variant of PCR where simultaneous amplification of more than one target nucleic acid sequence in one reaction vessel and subsequent or concurrent detection of the multiple products can be accomplished by using more than one pair of primers in a set (e.g., at least more than one forward and/or more than one reverse primer). Multiplex amplification can be useful not only for detecting the presence of a plurality of targets but also for the analysis, detection, and/or genotyping of deletions, mutations, and polymorphisms, and/or expression level and/or for quantitative assays. Multiplex can refer to the detection of between 2-1,000 different target sequences and/or alterations of a target nucleic acid in a single reaction. As used herein, multiplex refers to the detection of any range between 2-1,000, e.g., between 5-500, 25-1000, or 10-100 different target sequences in a single reaction, etc. By way of non-limiting example, a multiplex PCR reaction as part of a method described herein can affirmatively detect the presence of two or more possible alleles of at least two SNPs at at least two different allelic target site loci in a single reaction. The term “multiplex” as applied to PCR implies that there are primers specific for at least two different target sequences in the same PCR reaction. Thus, a reaction in which there are primer sets specific for two different target sequences is considered a multiplex amplification even if only one (or even none) of the at least two target sequences is actually detected in a given sample. Thus, in some embodiments, multiplex PCR can also refer to a reaction containing multiple pairs of primers, wherein the reaction can result in one or multiple specific amplified products when one or multiple targets are present in the reaction.

In some embodiments, the methods and compositions described herein relate to multimodal PCR. As used herein, “multimodal” refers to a variant of multiplex PCR where simultaneous amplification of more than one type or class of molecule or alteration occurs in one reaction vessel. Multimodal amplification can be useful for analysis of gene copy number, expression level, and/or sequence variation in some embodiments. Multimodal can refer to the detection of at least two different types of targets, i.e. 2 different types of targets, or 3 different types of targets. By way of non-limiting example, a multimodal PCR reaction can detect the level of gene copy number and the level of mRNA expression products in a single reaction, including quantitation of such targets.

Quantitative aspects can be facilitated, for example, by repeated sampling at any time during or after an amplification reaction, followed by separation and detection of the amplification products. Sampling can, for example, comprise removing an aliquot of the reaction. Sampling can occur, for example, at the end of every cycle, or at the end of every several cycles, e.g. every two cycles, every three cycles, every four cycles etc. While a uniform sample interval will most often be desired, there is no requirement that sampling be performed at uniform intervals. As just one example, the sampling routine can involve sampling after every cycle for the first five cycles, and then sampling after every other cycle or vice versa.

Sampling or dispensing of an aliquot from an amplification reaction can be performed in any of several different general formats. The sampling or removal method can depend on any of a number of factors including, but not limited to, the equipment available, the number of samples to be analyzed, and the timing of detection relative to sample collection (e.g., concurrently vs. sequential). The exact method of removal or extrusion of samples is not necessarily a limitation of the methods described herein. Sampling is preferably performed with an automated device, especially for high throughput applications. Sampling can also be performed using direct electrokinetic or hydrodynamic injection from a PCR reaction into a capillary electrophoretic device. The method of sampling used in the methods is preferably adapted to minimize contamination of the cycling reaction(s), by, for example, using pipetting tips or needles that are either disposed of after a single aliquot is withdrawn, or by using the same tip or needle for dispensing the sample from the same PCR reaction vessel. Methods for simultaneous sampling and detection are known to those skilled in the art (see, e.g., US Patent Application Publication 2004/0166513, incorporated herein by reference).

The amount of nucleic acid and/or volume of an aliquot dispensed at the sampling step can vary, depending, for example, upon the total volume of the amplification reaction, the sensitivity of product detection, and the type of sampling and/or separation used. Amplification volumes can vary from several microliters to several hundred microliters (e.g., 5 μl, 10 μl, 20 μl, 40 μl, 60 μl, 80 μl, 100 μl, 120 μl, 150 μl, or 200 μl or more), preferably in the range of 10-150 μl, more preferably in the range of 10-100 μl. The exact volume of the amplification reaction is not a limitation of the invention. Aliquot volumes can vary from 0.01% to 30% of the reaction mixture. Electrokinetic injection into capillary electrophoresis capillaries will generally load nucleic acid but not appreciably diminish the volume of the sampled reaction. The amplification regimen can be performed on plural independent nucleic acid amplification mixtures, optionally in a multiwell container. The container(s) in which the amplification reaction(s) are preformed is not necessarily a limitation of the methods described herein.

In various embodiments, the methods and compositions described herein relate to detecting amplified products (e.g. amplicons) for each target nucleic acid sequence, e.g. for each target alteration. In some embodiments, the detecting of the amplified product for each target nucleic acid sequence affirmatively indicates the presence of the target nucleic acid sequence in a sample. In some embodiments, the quantitative detecting of the amplified product for each target nucleic acid sequence indicates the level of that target nucleic acid sequence in a sample.

In some embodiments, the methods and compositions described herein relate to the amplified products of two or more primer pair subsets which should be distinguishable from each other. In some embodiments, the methods and compositions described herein relate to PCR amplification regimens wherein the amplified products of two or more primer pair subsets can be distinguished by being of distinct sizes. As used herein, a nucleic acid is of a “distinct size” if it is resolvable from nucleic acids of a different size. “Different sizes” refers to nucleic acid molecules that differ by at least one nucleotide in length. Generally, distinctly sized amplification products useful according to the methods described herein differ by a number of nucleotides greater than or equal to the limit of resolution for the separation process used in a given separation or detection method. For example, when the limit of resolution of separation is one base, distinctly sized amplification products differ by at least one base in length, but can differ by 2 bases, 5 bases, 10 bases, 20 bases, 50 bases, 100 bases or more. When the limit of resolution is, for example, 10 bases, distinctly sized amplification products will differ by at least 10 bases, but can differ by 11 bases, 15 bases, 20 bases, 30 bases, 50 bases, 100 bases or more.

In some embodiments, both the lengths of the primers or any portion thereof and the lengths of the segment of the target nucleic acid sequence that they anneal to can vary. Variation in the length of target sequence amplified, e.g. by chosen placement of the forward and reverse primers further or closer apart, is a straightforward approach to ensuring ready distinctions between products from different targets. Variation in the length of the primer, especially the 5′ tail regions of dual domain primers, is particularly effective, e.g. distinguishing the products of specific alleles of a given target locus in an assay.

In some embodiments the amplified products are distinguished by being labeled with different detectable labels. In some embodiments, the label is incorporated into a primer. In some embodiments, the label is conjugated to a primer.

In some embodiments, the label is bound to the primer after the PCR amplification regimen is complete. In some embodiments, the label is conjugated to an oligonucleotide or antibody or portion thereof that specifically binds to primer, or to a moiety attached thereto.

Two detectable labels are considered different if the signal from one label can be distinguished from the signal from the other. Detectable labels can comprise, for example, a light-absorbing dye, a fluorescent dye, or a radioactive label. Fluorescent dyes are preferred. Generally, a fluorescent signal is distinguishable from another fluorescent signal if the peak emission wavelengths are separated by at least 20 nm Greater peak separation is preferred, especially where the emission peaks of fluorophores in a given reaction are wide, as opposed to narrow or more abrupt peaks.

Detectable labels, methods of detecting them, and methods of incorporating them into or coupling and/or binding them to an amplified product are well known in the art. The following is provided by way of non-limiting example.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means.

The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies).

The detectable label can be linked by covalent or non-covalent means to nucleic acids. Alternatively, a detectable label can be linked such as by directly labeling a molecule that achieves binding to another nucleic acid via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In some embodiments, a detectable label can be a fluorescent dye molecule, or fluorophore including, but not limited to fluorescein, phycoerytllrin, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes.

In some embodiments, a detectable label can be a radiolabel including, but not limited to ³H, ¹²⁵I, ³⁵S, ¹⁴C, ³²P, and ³³P.

In some embodiments, a detectable label can be an enzyme including, but not limited to horseradish peroxidase and alkaline phosphatase. An enzymatic label can produce, for example, a chemiluminescent signal, a color signal, or a fluorescent signal.

In some embodiments, a detectable label is a chemiluminescent label, including, but not limited to luminol, luciferin or lucigenin.

In some embodiments, a detectable label can be a spectral colorimetric label including, but not limited to colloidal gold or colored glass or plastic (e.g., polystyrene, polypropylene, and latex) beads.

In some embodiments, the methods and compositions described herein relate to PCR amplification regimens wherein the amplified products of two or more primer pair subsets can be distinguished by being sequenced. Methods of sequencing nucleic acids are well known in the art and commercial sequencing services are widely available (e.g. Genscript; Piscataway, N.J.).

In some embodiments, the methods and compositions described herein relate to PCR amplification regimens wherein the amplified products of two or more primer pair subsets can be distinguished by melting-curve analysis. Methods of melting-curve analyses are well known in the art (e.g. Ririe et al. Analytical Biochemistry 1997 245:154-160; Wittwer et al. Clinical Chemistry 2003 49:853-860; and Liew et al. Clinical Chemistry 2007 50:1156-1164; which are incorporated by reference herein in their entireties).

Direct detection of size-separated amplification products is preferred. However, in some embodiments, the methods and compositions described herein relate to PCR amplification regimens wherein the amplified products of two or more primer pair subsets can be distinguished by oligonucleotide hybridization. One having ordinary skill in the art, using the sequence information of the target nucleic acid sequences, can design probes which are fully complementary to a single target and not to other target nucleic acid sequences. Hybridization conditions can be routinely optimized to minimize background signal by non-fully complementary hybridization. Hybridization probes can be designed to hybridize to the primer sequence, or part of the amplified product not comprised by the primer, provided that the sequence to which the probe will hybridize distinguishes it from at least one other amplified product present in the reaction.

In some embodiments, the PCR amplification regimen described herein is a multiplex and/or multimodal regimen. In some embodiments, an amplification product of one primer pair subset can be distinguished from the amplification products of other primer pair subsets by at least two approaches. By way of non-limiting example, all the products of a set of primers which amplify gDNA-specific targets of cMET can be labeled with one common label and each unique amplification product can be distinguished from the other amplification products of the same set of primers by being of a distinct size.

The methods and compositions described herein relate to the detection of the presence and/or level of a target nucleic acid sequence, e.g. the presence and/or level of a gene alteration in a sample. A target nucleic acid can be an RNA or a DNA. A target nucleic acid can be a double-stranded (ds) nucleic acid or a single-stranded (ss) nucleic acid, e.g. a dsRNA, a ssRNA, a dsDNA, or a ssDNA. As noted herein, it is specifically contemplated that methods described herein permit the detection and/or quantitation of more than one of these types of target in the same reaction, i.e. multimodal amplification and detection. Non-limiting examples of target nucleic acids include a nucleic acid sequence, a nucleic acid sequence comprising a mutation, a nucleic acid sequence comprising a deletion, a nucleic acid sequence comprising an insertion, a sequence variant, an allele, a polymorphism, a point mutation, a SNP, a microRNA, a protein coding RNA, a non-protein coding RNA, an mRNA, a nucleic acid from a pathogen (e.g. a bacterium, a virus, or a parasite), a nucleic acid associated with a disease or a likelihood of having or developing a disease (e.g. a marker gene, a polymorphism associated with a disease or a likelihood of having or developing a disease, or an RNA, the expression of which is associated with a disease or a likelihood of having or developing a disease).

A sample useful herein will comprise nucleic acids. In some embodiments, a sample can further comprise proteins, cells, fluids, biological fluids, preservatives, and/or other substances. In some embodiments, a sample can be obtained from a subject. In some embodiments, a sample can be a biological sample obtained from the subject. In some embodiments a sample can be a diagnostic sample obtained from a subject. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample is a clarified fluid sample, for example, by centrifugation. In some embodiments, the sample is clarified by low-speed centrifugation (e.g. 3,000×g or less) and collection of the supernatant comprising the clarified fluid sample.

In some embodiments, the sample can be freshly collected. In some embodiments, the sample can be stored prior to being used in the methods and compositions described herein. In some embodiments, the sample is an untreated sample. As used herein, “untreated sample” refers to a biological sample that has not had any prior sample pre-treatment except for dilution and/or suspension in a solution.

In some embodiments, a sample can be obtained from a subject and preserved or processed prior to being utilized in the methods and compositions described herein. By way of non-limiting example, a sample can be embedded in paraffin wax, refrigerated, or frozen. A frozen sample can be thawed before determining the presence of a nucleic acid according to the methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g. anti-coagulant or nuclease inhibitor) and any combination thereof. In some embodiments, the sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in the methods and compositions described herein. The skilled artisan is well aware of methods and processes for processing, preservation, or treatment of samples for nucleic acid analysis.

In some embodiments, the nucleic acid sample can be prepared from a FFPE tumor sample. In some embodiments, the sample can comprise tumor cels from a subject having, or diagnosed as having gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and/or thyroid cancer. See, e.g. Sattler et al. Ther Adv Med Oncol 2011 3:171-184; which is incorporated by reference herein in its entirety.

In some embodiments, the nucleic acid present in a sample is isolated, enriched, or purified prior to being utilized in the methods and compositions described herein. Methods of isolating, enriching, or purifying nucleic acids from a sample are well known to one of ordinary skill in the art. By way of non-limiting example, kits for isolation of genomic DNA from various sample types are commercially available (e.g. Catalog Nos. 51104, 51304, 56504, and 56404; Qiagen; Germantown, Md.).

The terms “subject” and “individual” are used interchangeably herein, and refer to an organism from which a sample is obtained. A subject can be any organism for which it is desired to determine the presence of a nucleic acid in the organism or one or more cells comprising or contained within that organism. As used herein, a “subject” can mean an organism, e.g. a bacterium, a parasite, a plant, or an animal. In some embodiments, a subject can be a human or animal. Usually the animal is a vertebrate such as a primate, rodent, domestic animal or game animal. Primates include chimpanzees, cynomologous monkeys, spider monkeys, and macaques, e.g., Rhesus monkeys. Rodents include, e.g., mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Individual or subject includes any subset of the foregoing, e.g., all of the above.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, “altered” can refer to, e.g. a statistically significant change in a level or number (e.g. gene expression level or gene copy number) relative to a reference or a change in a sequence, e.g. at least a single nucleotide change in a nucleic acid sequence relative to a reference.

As used herein, “normalize” refers to a process of dividing a first value by a second value, e.g. obtaining a level of x per level of y. X is typically the thing being measured, e.g. copy number or expression level of cMet, while y is a reference, e.g. the copy number or expression level of a reference gene. Normalization allows the levels measured in multiple samples and/or reactions to be compared by controlling for, e.g. the level of nucleic acid present in the samples as well as differing efficiencies between reactions. The selection of reference genes and preferred means of normalizing different values are described elsewhere herein.

As used herein, a “portion” refers to a part or fraction of a whole, e.g. a part or fraction of a total molecule. A particular molecule can have multiple portions, e.g. two portions, three portions, four portions, five portions, or more portions.

The term “isolated” or “partially purified” as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered “isolated.”

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand of a denatured double-stranded DNA. Alternatively, it can be a single-stranded nucleic acid not derived from any double-stranded DNA. In one aspect, a template nucleic acid is DNA. In another aspect, a template is RNA. Suitable nucleic acid molecules include DNA, including genomic DNA and cDNA. Other suitable nucleic acid molecules include RNA, including mRNA, rRNA and tRNA. The nucleic acid molecule can be naturally occurring, as in genomic DNA, or it may be synthetic, i.e., prepared based upon human action, or may be a combination of the two. The nucleic acid molecule can also have certain modifications such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleosides that are linked between the 2′-oxygen and the 4′-carbon atoms with a methylene unit as described in U.S. Pat. No. 6,268,490, wherein both patent and patent application are incorporated herein by reference in their entirety.

The term “gene” means a nucleic acid sequence which is transcribed (DNA) to RNA in vitro or in vivo when operably linked to appropriate regulatory sequences. The gene can include regulatory regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complementary” refers to the hierarchy of hydrogen-bonded base pair formation preferences between the nucleotide bases G, A, T, C and U, such that when two given polynucleotides or polynucleotide sequences anneal to each other, A pairs with T and U pairs with C in DNA, and G pairs with C and A pairs with U in RNA. As used herein, “substantially complementary” refers to a primer having at least 90% complementarity over the entire length of a primer with a second nucleotide sequence, e.g. 90% complementary, 95% complementary, 98% complementary, 99% complementary, or 100% complementary.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); and Kendrew et al. (eds.), Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An assay for detecting cMET alterations, the assay comprising
   contacting a portion of a nucleic acid sample with two sets of primers wherein the first set of primers detects alterations in cMET gene copy number variation and the second set of primers detects changes in cMET gene expression level;
   wherein the first set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation;
   wherein the second set of primers comprises subsets of primer pairs that amplify mRNA-specific sequences of cMET and mRNA-specific sequences of at least two reference genes;
   performing a PCR amplification regimen comprising cycles of strand separation, primer annealing, and primer extension on a reaction mixture comprising the portion of the sample and the two sets of primers;
   detecting the level of the amplicon for each primer pair;
   normalizing the level of cMET amplicons to the reference gene amplicons;
   and comparing the normalized level of cMET amplicons to a reference level; wherein a higher level of a gDNA-specific cMET amplicon as compared to the reference level indicates the presence of a gene amplification alteration of cMET in the sample, and an altered level of a mRNA-specific cMET amplicon as compared to the reference level indicates the presence of a gene expression level alteration of cMET in the sample.
2. The assay of paragraph 1, wherein the first set of primers further comprises a subset of primer pairs that amplify at least one gDNA-specific sequence of EGFR; and
   the assay further comprises comparing the normalized level of EGFR amplicons to a reference level; wherein a higher level of a gDNA-specific EGFR amplicon as compared to the reference level indicates the presence of a gene amplification alteration of EGFR in the sample.
3. The assay of any of paragraphs 1-2, wherein the reference gene of the first primer set which is located on chromosome 7 is KDELR-2; and
   the assay further comprises comparing the normalized level of KDELR-2 amplicons to a reference level; wherein a higher level of a gDNA-specific KDELR-2 amplicon as compared to the reference level indicates the presence of a gene amplification alteration of KDELR-2 in the sample.
4. The assay of any of paragraphs 1-3, wherein the presence of a gene amplification alteration of cMET, EGFR and KDELR-2 indicates the presence of chromosome 7 amplification.
5. The assay of paragraphs 1-4, wherein the reference gene of the first primer set which is not located on chromosome 7 is SOD1 or SPG21.
6. The assay of paragraph 5, wherein the first primer set comprises subsets of primer pairs that amplify at least one gDNA-specific sequence of each of SOD1 and SPG21.
7. The assay of any of paragraphs 1-6, wherein a primer set comprises primer pair subsets that amplify at least one amplicon of each gene.
8. The assay of any of paragraphs 1-7, wherein a primer set comprises primer pair subsets that amplify at least two amplicons of each gene.
9. The assay of any of paragraphs 1-8, wherein a primer set comprises primer pair subsets that amplify at least three amplicons of each gene.
10. The assay of any of paragraphs 1-9, wherein the primer sets comprise primer pair subsets that amplify at least two gDNA-specific amplicons of each of cMET, EGFR, and KDELR-2 and at least two mRNA-specific amplicons of each of cMET, SOD1 and SGP21.
11. The assay of any of paragraphs 1-10, wherein the primer sets comprise primer pair subsets that amplify at least three gDNA-specific amplicons of each of cMET, EGFR, and KDELR-2 and at least three mRNA-specific amplicons of each of cMET, SOD1 and SGP21.
12. The assay of any of paragraphs 1-11, further comprising:
   contacting a second portion of the sample with a third set of primer pairs wherein the third set of primers comprises subsets of primer pairs that amplify cMET sequences comprising sequence variations;
   performing a PCR amplification regimen comprising cycles of strand separation, primer annealing, and primer extension on a reaction mixture comprising the second portion of the sample and the third set of primers;
   detecting the level of the amplicon for each primer pair, wherein the presence of an amplicon indicates the presence of the sequence variation for which that primer pair is specific.
13. The assay of paragraph 12, wherein one or more sequence variations of cMET are SNPs.
14. The assay of any of paragraphs 12-13, wherein the cMET SNP is selected from the group consisting of:
   S1058P; V1101I; H1112Y; H1124D; G1137V; M1149T; V1206L; L1213V; K1262R; M1268T; V12381; Y1248C; and D1246N.
15. The assay of any of paragraphs 12-14, wherein S1058P; V1101I; H1112Y; H1124D; G1137V; M1149T; V1206L; L1213V; K1262R; M1268T; V12381; Y1248C; and D1246N are detected.
16. The assay of any of paragraphs 12-15, wherein the same PCR thermocycling regimens are used for both reactions.
17. The assay of any of paragraphs 1-16, wherein the nucleic acid sample is prepared from a FFPE tumor sample.
18. The assay of any of paragraphs 1-17, wherein the sample comprises tumor cells from a subject diagnosed with a condition selected from the group consisting of:
   gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.
19. The assay of any of paragraphs 1-18, wherein one or more primers are dual domain primers.
20. The assay of any of paragraphs 1-19, wherein an amplified products from two or more primer pairs of a primer subset can be distinguished.
21. The assay of any of paragraphs 1-20, wherein the amplified products from two or more primer pairs of a primer subset are distinguished by being of distinct sizes.
22. The assay of any of paragraphs 1-21, wherein the amplified products from two or more primer pairs of a primer subset are distinguished by being labeled with different detectable labels.
23. The assay of any of paragraphs 1-22, wherein the amplified products from the first set of primers and the second set of primers are distinguished by being labeled with different detectable labels.
24. The assay of any of paragraphs 1-23, wherein one or more primers are selected from the group consisting of SEQ ID NOs: 1-83.
25. The assay of any of paragraphs 1-24, wherein one or more primers comprise a sequence of any of SEQ ID NOs: 89-124.
26. The assay of any of paragraphs 1-25, wherein the primers are present in the reaction mixture at about the concentrations of Table 2.
27. A method of detecting cMET alterations, the method comprising
   contacting a portion of a nucleic acid sample with a set of primers which detect alterations in cMET gene copy number variation;
   wherein the set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation;
   performing a PCR amplification regimen comprising cycles of strand separation, primer annealing, and primer extension on a reaction mixture comprising the portion of the sample and the set of primers;
   detecting the level of the amplicon for each primer pair;
   normalizing the level of cMET amplicons to the reference gene amplicons;
   and comparing the normalized level of cMET amplicons to a reference level; wherein a higher level of a gDNA-specific cMET amplicon as compared to the reference level indicates the presence of a gene amplification alteration of cMET in the sample.
28. The method of paragraph 27, wherein the set of primers further comprises a subset of primer pairs that amplify at least one gDNA-specific sequence of EGFR; and
   the assay further comprises comparing the normalized level of EGFR amplicons to a reference level; wherein a higher level of a gDNA-specific EGFR amplicon as compared to the reference level indicates the presence of a gene amplification alteration of EGFR in the sample.
29. The method of any of paragraphs 27-28, wherein the reference gene of the primer set which is located on chromosome 7 is KDELR-2; and
   the method further comprises comparing the normalized level of KDELR-2 amplicons to a reference level; wherein a higher level of a gDNA-specific KDELR-2 amplicon as compared to the reference level indicates the presence of a gene amplification alteration of KDELR-2 in the sample.
30. The method of any of paragraphs 27-29, wherein the presence of a gene amplification alteration of cMET, EGFR and KDELR-2 indicates the presence of chromosome 7 amplification.
31. The method of paragraphs 27-30, wherein the reference gene of the primer set which is not located on chromosome 7 is SOD1 or SPG21.
32. The method of paragraph 31, wherein the primer set comprises subsets of primer pairs that amplify at least one gDNA-specific sequence of SOD1 and SPG21.
33. The method of any of paragraphs 27-32, further comprising contacting the portion of a nucleic acid sample with a second set of primers, wherein the second set of primers detects changes in cMET gene expression level;
   wherein the second set of primers comprises subsets of primer pairs that amplify mRNA-specific sequences of cMET and at least mRNA specific sequences of at least two reference genes; and
   wherein an altered level of a mRNA-specific cMET amplicon as compared to the reference level indicates the presence of a gene expression level alteration of cMET in the sample.
34. The method of any of paragraphs 27-33, wherein the first primer set comprises subsets of primer pairs that amplify at least one gDNA-specific sequence of each of SOD1 and SPG21.
35. The method of any of paragraphs 27-34, wherein a primer set comprises primer pair subsets that amplify at least one amplicon of each gene.
36. The method of any of paragraphs 27-35, wherein a primer set comprises primer pair subsets that amplify at least two amplicons of each gene.
37. The method of any of paragraphs 27-36, wherein a primer set comprises primer pair subsets that amplify at least three amplicons of each gene.
38. The method of any of paragraphs 27-37, wherein the primer sets comprise primer pair subsets that amplify at least two gDNA-specific amplicons of each of cMET, EGFR, and KDELR-2 and at least two mRNA-specific amplicons of each of cMET, SOD1 and SGP21.
39. The method of any of paragraphs 27-38, wherein the primer sets comprise primer pair subsets that amplify at least three gDNA-specific amplicons of each of cMET, EGFR, and KDELR-2 and at least three mRNA-specific amplicons of each of cMET, SOD1 and SGP21.
40. The method of any of paragraphs 27-39, further comprising:
   contacting a second portion of the sample with a third set of primer pairs wherein the third set of primers comprises subsets of primer pairs that amplify cMET sequences comprising sequence variations;
   performing a PCR amplification regimen comprising cycles of strand separation, primer annealing, and primer extension on a reaction mixture comprising the second portion of the sample and the third set of primers;
   detecting the level of the amplicon for each primer pair, wherein the presence of an amplicon indicates the presence of the sequence variation for which that primer pair is specific.
41. The method of paragraph 40, wherein one or more sequence variations of cMET are SNPs.
42. The method of any of paragraphs 39-41, wherein the cMET SNP is selected from the group consisting of:
   S1058P; V1101I; H1112Y; H1124D; G1137V; M1149T; V1206L; L1213V; K1262R; M1268T; V12381; Y1248C; and D1246N.
43. The method of any of paragraphs 39-42, wherein S1058P; V1101I; H1112Y; H1124D; G1137V; M1149T; V1206L; L1213V; K1262R; M1268T; V12381; Y1248C; and D1246N are detected.
44. The method of any of paragraphs 39-43, wherein the same PCR thermocycling regimens are used for both reactions.
45. The method of any of paragraphs 39-44, wherein the nucleic acid sample is prepared from a FFPE tumor sample.
46. The method of any of paragraphs 27-45, wherein the sample comprises tumor cells from a subject diagnosed with a condition selected from the group consisting of:
   gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.
47. The method of any of paragraphs 27-46, wherein one or more primers are dual domain primers.
48. The method of any of paragraphs 27-47, wherein an amplified products from two or more primer pairs of a primer subset can be distinguished.
49. The method of any of paragraphs 27-48, wherein the amplified products from two or more primer pairs of a primer subset are distinguished by being of distinct sizes.
50. The method of any of paragraphs 27-49, wherein the amplified products from two or more primer pairs of a primer subset are distinguished by being labeled with different detectable labels.
51. The method of any of paragraphs 27-50, wherein the amplified products from the first set of primers and the second set of primers are distinguished by being labeled with different detectable labels.
52. The method of any of paragraphs 27-51, wherein one or more primers are selected from the group consisting of SEQ ID NOs: 1-83.
53. The method of any of paragraphs 27-52, wherein one or more primers comprise a sequence of any of SEQ ID NOs: 89-124.
54. The method of any of paragraphs 27-53, wherein the primers are present in the reaction mixture at about the concentrations of Table 2.

### EXAMPLES

### Example 1

An 18-target, single-tube multimodal assay designed to detected amplification of cMET and EGFR genes, expression of cMET and polysomy of chromosome 7 compatible with the ICEPlex system was developed. The assay was tested using cell lines previously characterized in the literature.

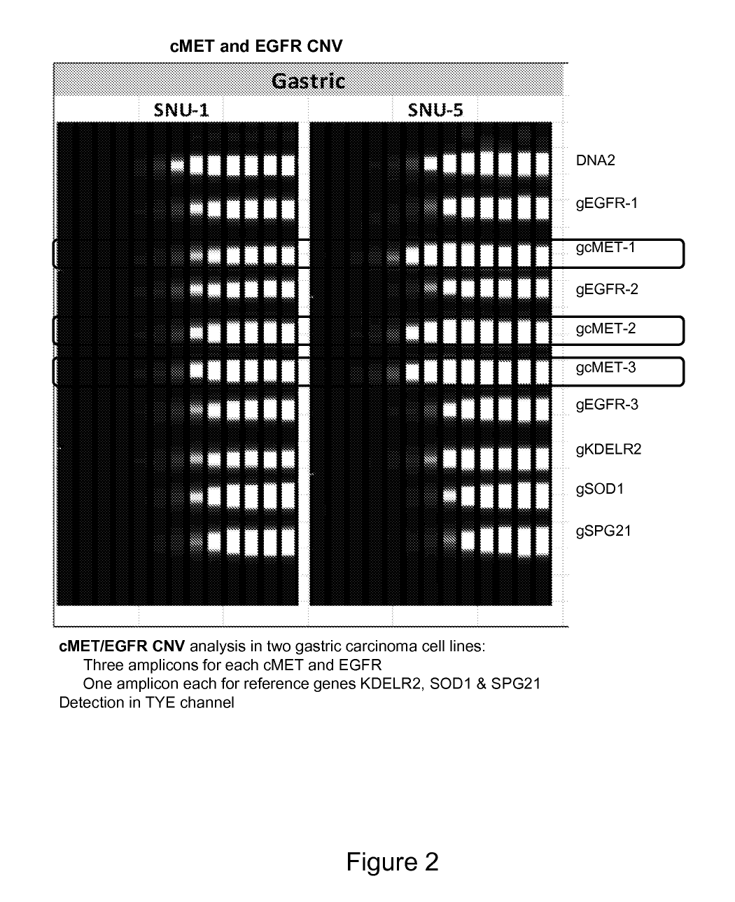

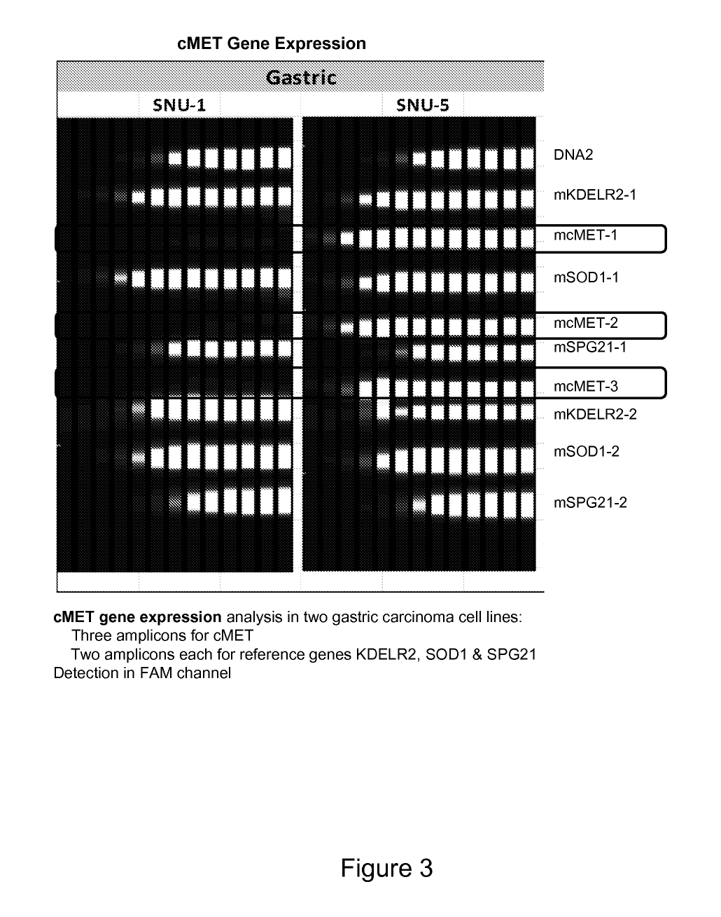

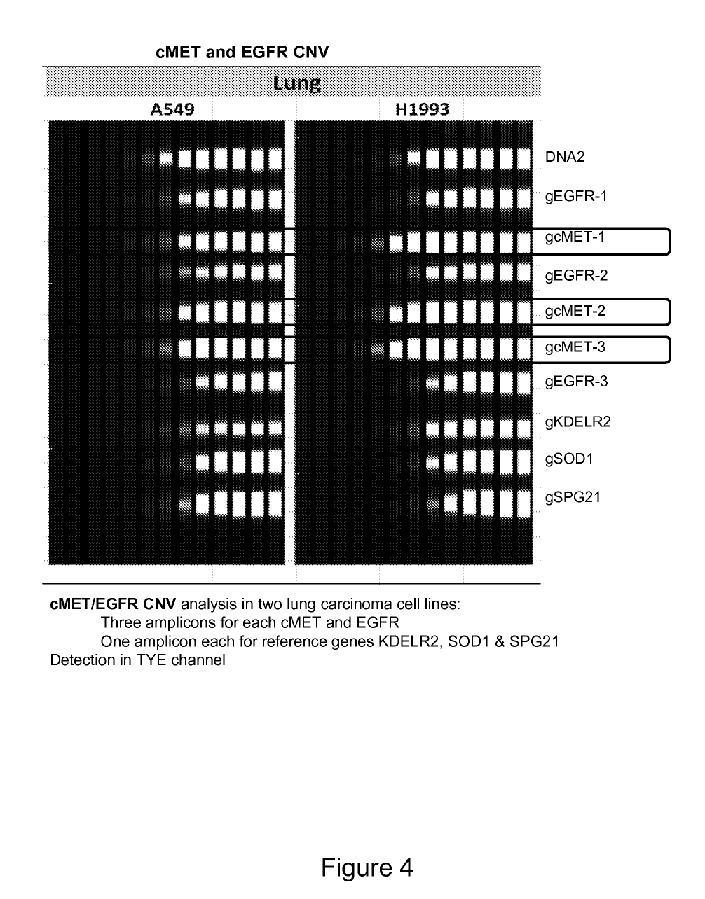

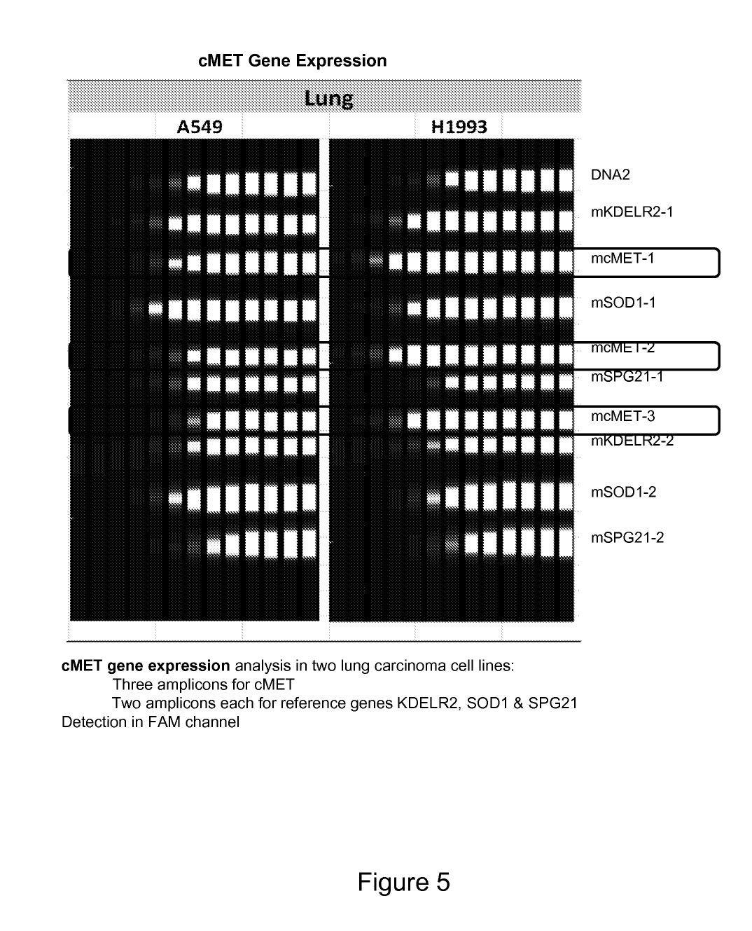

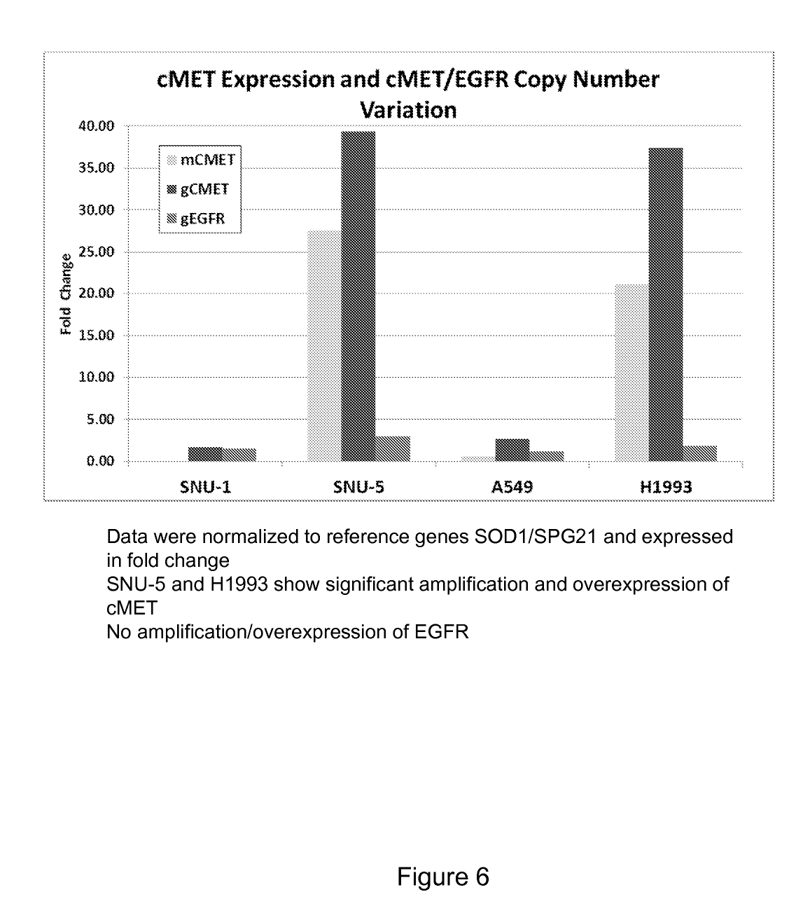

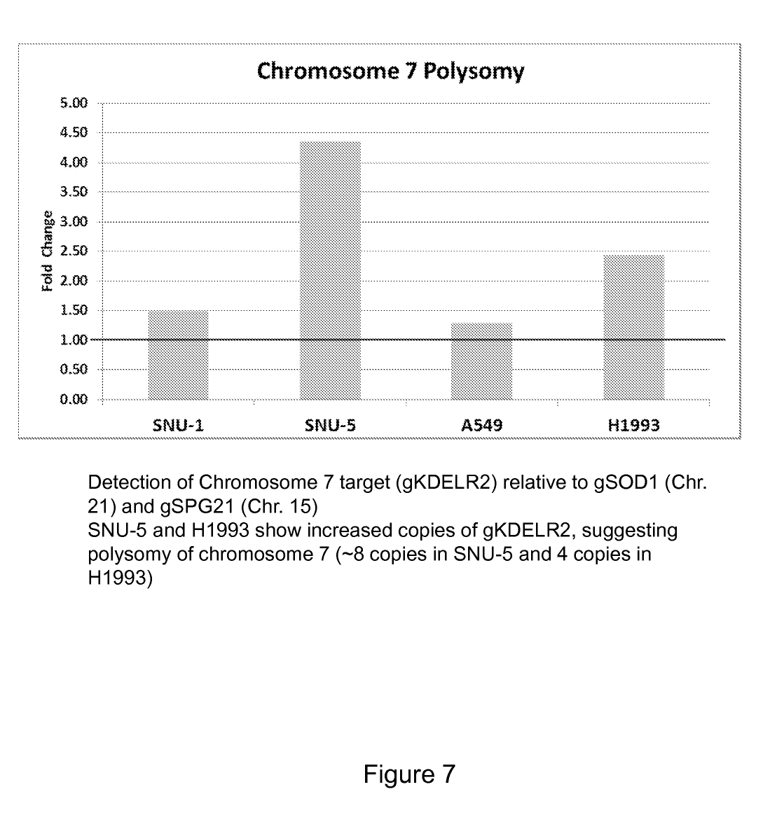

Amplification of cMET is known to be present in cell lines SNU-5 and H1993. Overexpression of cMET is known to occur in SNU-5 and no expression of cMET has been reported in SNU-1. Chromosome 7 polysomy is known to exist for cell lines SNU-5 and possibly for H1993. The assay was performed with the primers of Table 1 using the concentrations shown in Table 2 and confirmed the prior characterization of the cell lines (Table 5), as depicted in FIGS. 2-7.

Further, the assay described herein revealed no abnormal levels of cMET or chromosome 7 polysomy in normal tissue or a single clinical FFPE specimen (data not shown). When the assay was tested on normal lung and gastric tissue or on clinical FFPE gastric cancer specimen no abnormal status of cMET, EGFR or chromosome 7 was revealed (data not shown).

Suitable buffers can include the following: Tris buffer (50-200 mM, pH 8-9), Trehalose (5-15%), Potassium Acetate (25-150 mM), Glycerol (1-7.5%), and betaine (250-1250 mM). delta-exo-Apta Taq Polymerase was used (1-10 U per PCR reaction). Thermocycling conditions are depicted in FIG. 11.

TABLE 1
Primer Sequences

| | | | | | SEQ ID |
|---|---|---|---|---|---|
| Primer | Target | Label | Sequence | Bases | NO |
| RT Primers | | | | | |
| cMET_e14e15_RT1 | mM1 | | GTC TGT CAG AGG ATA | 17 | 1 |
| | | | CT | | |
| cMET_e5e6_RT1 | mM3 | | TTG TCC CTC CTT CAA G | 16 | 2 |
| cMET_e8e9_RT1 | mM2 | | GCT GGG GTA TAA CAT | 17 | 3 |
| | | | TC | | |
| mKDELR2- | mKDEL-2 | | AAA AAG ATC CAG GTA | 18 | 4 |
| 1_RT1 | (‘KDEL’ | | ACG | | |
| | disclosed | | | | |
| | as | | | | |
| | SEQ ID | | | | |
| | NO: | | | | |
| | 130) | | | | |
| mKDELR2- | mKDEL-1 | | TTT CAG GTA GAT CAG | 17 | 5 |
| 2_RT1 | (‘KDEL’ | | GT | | |
| | disclosed | | | | |
| | as | | | | |
| | SEQ ID | | | | |
| | NO: | | | | |
| | 130) | | | | |
| mSOD1-1_RT1 | mSOD-2 | | AGA GGA TTA AAG TGA | 18 | 6 |
| | | | GGA | | |
| mSOD1-2_RT1 | mSOD-1 | | ACT TTC TTC ATT TCC | 18 | 7 |
| | | | ACC | | |
| mSPG21-1_RT1 | mSPG-2 | | GCC AGA TGA AAA ATT | 18 | 8 |
| | | | TCC | | |
| mSPG21-2_RT1 | mSPG-1 | | CAT GGA ATT GCA GCA A | 16 | 9 |
| Forward Primers | | | | | |
| gCMET_e2-I- | gM1 | | AAC GCC CGC TTT ATT | 42 | 10 |
| e3_F1-MTA1 | | | AAT ATT CTA TGT TCT | | |
| | | | TAT CTC CTC AGT | | |
| gCMET_e3-I- | gM2 | | TGA GTT ACC ATT AAA | 47 | 11 |
| e4_F2-MTA3 | | | ATA ATA AAT TAA TTG | | |
| | | | GTT CCA TCC TAG CTC | | |
| | | | TT | | |
| gCMET_e5-I-e6 | gM3 | | AGC TTA AAC GAA ATA | 49 | 12 |
| F2-MTA2 | | | TTA AAT ATT ATT ATT | | |
| | | | AAC TCA CCC ACT CTC | | |
| | | | TGA T | | |
| gEGFR_e1-I- | gE2 | | TCA GAA GGA CAA TAT | 38 | 13 |
| e2_2 F1-MTA1 | | | TTT TAC CCA GTG ACT | | |
| | | | TAC CTA TG | | |
| gEGFR_e1-I- | gE3 | | TAT CGT AAC ATA ATT | 58 | 14 |
| e2_F1-MTA2 | | | TAA TAA TAA AAT AAT | | |
| | | | TTA ATT ATT TCA AAT | | |
| | | | CTG GAA AGG ACA C | | |
| gEGFR_e3-I- | gE1 | | TCC TGC GCT GTA TAA | 34 | 15 |
| e4_F2-MTA1 | | | ACT TCT GGG GAA GCT | | |
| | | | CAT T | | |
| gKDELR2_e1_I_e2_F3- | gKDEL | | ACT TTG CCT AAA TAA | 53 | 16 |
| MTA2 | (‘KDEL’ | | ATA TTA ATA ATT AAT | | |
| | disclosed | | ATA TCA GCA TCT GAA | | |
| | as | | ACC CAT AG | | |
| | SEQ ID | | | | |
| | NO: | | | | |
| | 130) | | | | |
| gSOD1_e2_I_e3_F2- | gSOD | | TAA ACT CCC TAT AAA | 60 | 17 |
| MTA2 | | | ATT AAA TTA ATA ATA | | |
| | | | TAT AAT ATT TTG TGC | | |
| | | | TCT GTG AAT GTC ATC | | |
| gSPG21_e7_I_e9_F2- | gSPG | | TGT GGA GAT TAT AAA | 60 | 18 |
| MTA2 | | | ATT AAT TAA TAA TAT | | |
| | | | ATA ATA TTT TTA CCC | | |
| | | | AGG TTT CCA GAA TAG | | |
| mCMET_e14e15_F1- | mM1 | | AAG CTT CGT GAT AAT | 39 | 19 |
| MTA11 | | | TAA ATC TGT AGA CTA | | |
| | | | CCG AGC TAC | | |
| mCMET_e5e6_F2- | mM3 | | TAG GAT GGC CTA TTT | 54 | 20 |
| MTA1 | | | TAA TAA AAT AAT TTT | | |
| | | | ATA ATT AAT CGG AGG | | |
| | | | AAT GCC TGA | | |
| mCMET_e8e9_F1- | mM2 | | AGA AGG ACC GTT TTA | 51 | 21 |
| MTA11 | | | TTT ATT TTA TTA TAC | | |
| | | | TAA ACA GTG GGA ATT | | |
| | | | CTA GAC | | |
| mKDELR2_e1_e2_F1- | mKDEL-2 | | TTG AGA TGG CAT TAA | 55 | 22 |
| MTA5 | (‘KDEL’ | | TTA AAT TTT TAA TAA | | |
| | disclosed | | TAT TTA CTG CTG AAG | | |
| | as | | ATC TGG AAG A | | |
| | SEQ ID | | | | |
| | NO: | | | | |
| | 130) | | | | |
| mKDELR2_e2_e3_F2- | mKDEL-1 | | ACT TTG CCT AAA TAT | 47 | 23 |
| MTA11 | (‘KDEL’ | | ATT TTT CTT CAT TTA | | |
| | disclosed | | TTT CAT TGT ATA ACA | | |
| | as | | CA | | |
| | SEQ ID | | | | |
| | NO: | | | | |
| | 130) | | | | |
| mSOD1_e2_e3_F1- | mSOD-2 | | ATC TAT ATA AAT AAT | 67 | 24 |
| MTA2 | | | TTT ATA AAA TAA TTT | | |
| | | | ATT AAA ATT AAA TAT | | |
| | | | ATG CAT TAA AGG ACT | | |
| | | | GAC TGA A | | |
| mSOD1_e4_e5_F1- | mSOD-1 | | ACC ATG GTT TAT AAT | 42 | 25 |
| MTA11 | | | AAA TAT TAA GAT CTC | | |
| | | | ACT CTC AGG AGA | | |
| mSPG21_e6_e7_F2- | mSPG-2 | | AAG CAG CAG ATA ATT | 75 | 26 |
| MTA2 | | | TAT TAT ATA ATT AAA | | |
| | | | AAT AAT TAT AAT TAA | | |
| | | | TAA AAT TTA AAC ACC | | |
| | | | TCT ATC TTC AAC CAA | | |
| mSPG21_e9_e10_F2- | mSPG-1 | | ACC ATC TCG GTA ATT | 57 | 27 |
| MTA2 | | | AAT AAT TAA AAT AAT | | |
| | | | TTA ATT ATG CTC ATC | | |
| | | | TGA AAA CAG GAG | | |
| Reverse Primers | | | | | |
| gCMET_e2-I- | gM1 | TYE | /5TYE665/TCA TTG CCC | 35 | 28 |
| e3_R1 TYE | | | TTT TAA ATA AGC AGT | | |
| | | | GGC AGA AAT TC | | |
| gCMET_e3-I- | gM2 | TYE | /5TYE665/AGC ATG CGT | 35 | 29 |
| e4_R2 TYE | | | ATT TAA GTT AAG AGG | | |
| | | | CAG AAG AGA AC | | |
| gCMET_e5-I-e6 | gM3 | TYE | /5TYE665/ATA GCT GTT | 34 | 30 |
| R2 TYE | | | ATT TAA CAG GAT ATG | | |
| | | | CCA TGA ACA G | | |
| gEGFR_e1-I- | gE2 | TYE | /5TYE665/ATG ATG GAG | 33 | 31 |
| e2_2 R2 TYE | | | TTT TAA CTG CCT GCT | | |
| | | | ACT GTA TGA | | |
| gEGFR_e1-I- | gE3 | TYE | /5TYE665/AGG CCA CCG | 35 | 32 |
| e2_R1 TYE | | | TTT TAA TGT TAA AAG | | |
| | | | CCT ATT GGA GC | | |
| gEGFR_e3-I- | gE1 | TYE | /5TYE665/TTC ATG CAA | 34 | 33 |
| e4_R2 TYE | | | TTT TAA CAT GTT GTG | | |
| | | | TGT ACA GAG T | | |
| gKDELR2_e1_I_e2_R3 | gKDEL | TYE | /5TYE665/AGG AGA AGT | 46 | 34 |
| TYE | | | CTT TTT ATA TTT ATT | | |
| | | | ATA TGG ACA TTT ATG | | |
| | | | TGG TGT G | | |
| gSOD1_e2_I_e3_R2 | gSOD | TYE | /5TYE665/ACT AGT TGC | 49 | 35 |
| TYE | | | TAT TAA TTA AAA TTT | | |
| | | | TTA TAT TTT GCT GCC | | |
| | | | TTA CAC AAC T | | |
| gSPG21_e7_I_e9_R2 | gSPG | TYE | /5TYE665/ACT AGT TGC | 54 | 36 |
| TYE | | | TAT TTA ATA ATA AAT | | |
| | | | TTA AAA ATA TCA GAA | | |
| | | | AAG TCA TCA GTG AGG | | |
| mCMET_e14e15_R1 | mM1 | FAM | /56-FAM/TTG CGA TCC | 34 | 37 |
| FAM | | | CTT TAA GTC TGT CAG | | |
| | | | AGG ATA CTG C | | |
| mCMET_e5e6_R2 | mM3 | FAM | /56-FAM/AAA CTT CGC | 32 | 38 |
| FAM | | | ATT TAA TTG TCC CTC | | |
| | | | CTT CAA GG | | |
| mCMET_e8e9_R1 | mM2 | FAM | /56-FAM/TCG CGC TAG | 35 | 39 |
| FAM | | | ATT TAA GCT GGG GTA | | |
| | | | TAA CAT TCA AG | | |
| mKDELR2_e1_e2_R1 | mKDEL-2 | FAM | /56-FAM/TTT ATG CCA | 46 | 40 |
| FAM | (‘KDEL’ | | TTT ATA ATA ATA TAA | | |
| | disclosed | | AAA AAA AGA TCC AGG | | |
| | as | | TAA CGA G | | |
| | SEQ ID | | | | |
| | NO: | | | | |
| | 130) | | | | |
| mKDELR2_e2_e3_R2 | mKDEL-1 | FAM | /56-FAM/AGG AGA AGT | 35 | 41 |
| FAM | (‘KDEL’ | | CTT TAA TTT CAG GTA | | |
| | disclosed | | GAT CAG GTA CA | | |
| | as | | | | |
| | SEQ ID | | | | |
| | NO: | | | | |
| | 130) | | | | |
| mSOD1_e2_e3_R1 | mSOD-2 | FAM | /56-FAM/TTC CGT AAA | 35 | 42 |
| FAM | | | CTT TAA AGA GGA TTA | | |
| | | | AAG TGA GGA CC | | |
| mSOD1_e4_e5_R1 | mSOD-1 | FAM | /56-FAM/AAC CAT ACG | 35 | 43 |
| FAM | | | ATT TAA ACT TTC TTC | | |
| | | | ATT TCC ACC TT | | |
| mSPG21_e6_e7_R2 | mSPG-2 | FAM | /56-FAM/TGC ATA AGA | 37 | 44 |
| FAM | | | ATT TAA TAG CCA GAT | | |
| | | | GAA AAA TTT CCA A | | |
| mSPG21_e9_e10_R2 | mSPG-1 | FAM | /56-FAM/AGG AGA AGT | 34 | 45 |
| FAM | | | CTT TAA CAT GGA ATT | | |
| | | | GCA GCA AAT G | | |

TABLE 2
Exemplary embodiment of multiplex primer pair sets and concentrations

| | Amp | | | For | Rev |
|---|---|---|---|---|---|
| Target | Size | Forward | Reverse | (uM) | (uM) |
| mM1 | 124 | mCMET_e14e15_F1-MTA11 | mCMET_e14e15_R1 FAM | 1.3 | 1.3 |
| mM2 | 135.5 | mCMET_e8e9_F1-MTA11 | mCMET_e8e9_R1 FAM | 1.6 | 1.6 |
| mM3 | 146.5 | mCMET_e5e6_F2-MTA1 | mCMET_e5e6_R2 FAM | 1.6 | 1.6 |
| gM1 | 127 | gCMET_e2-I-e3_F1-MTA1 | gCMET_e2-I-e3_R1 TYE | 2 | 2 |
| gM2 | 139 | gCMET_e3-I-e4_F2-MTA3 | gCMET_e3-I-e4_R2 TYE | 2.2 | 2.2 |
| gM3 | 144 | gCMET_e5-I-e6 F2-MTA2 | gCMET_e5-I-e6 R2 TYE | 1.8 | 1.8 |
| gE1 | 120 | gEGFR_e3-I-e4_F2-MTA1 | gEGFR_e3-I-e4_R2 TYE | 2.5 | 2.5 |
| gE2 | 132 | gEGFR_e1-I-e2_2 F1-MTA1 | gEGFR_e1-I-e2_2 R2 TYE | 4 | 4 |
| gE3 | 150 | gEGFR_e1-I-e2_F1-MTA1 | gEGFR_e1-I-e2_R1 TYE | 2.8 | 2.8 |
| mSPG2 | 165 | mSPG21_e6_e7_F2_MTA2 | mSPG21_e6_e7_R2 FAM | 2.5 | 2.5 |
| mSPG1 | 144 | mSPG21_e9_e10_F2_MTA2 | mSPG21_e9_e10_R2 FAM | 2.5 | 2.5 |
| gSPG | 169.5 | gSPG21_e7_I_e9_F2_MTA2 | gSPG21_e7_I_e9_R2 TYE | 3.8 | 3.8 |
| mKDEL2 | 151 | mKDELR2_e1_e2_F1_MTA5 | mKDELR2_e1_e2_R1 FAM | 1.6 | 1.6 |
| (‘KDEL’ | | | | | |
| disclosed | | | | | |
| as SEQ ID | | | | | |
| NO: 130) | | | | | |
| mKDEL1 | 118 | mKDELR2_e2_e3_F2_MTA11 | mKDELR2_e2_e3_R2 FAM | 1.5 | 1.5 |
| (‘KDEL’ | | | | | |
| disclosed | | | | | |
| as SEQ ID | | | | | |
| NO: 130) | | | | | |
| gKDELR | 157 | gKDELR2_e1_I_e2_F3_MTA2 | gKDELR2_e1_I_e2_R3 TYE | 4.5 | 4.5 |
| mSO2 | 158 | mSOD1_e2_e3_F1_MTA2 | mSOD1_e2_e3_R1 FAM | 2 | 2 |
| mSO1 | 130 | mSOD1_e4_e5_F1_MTA3 | mSOD1_e4_e5_R1 FAM | 1.8 | 1.8 |
| gSOD | 163 | gSOD1_e2_I_e3_F2_MTA2 | gSOD1_e2_I_e3_R2 TYE | 2.7 | 2.7 |

### Example 2

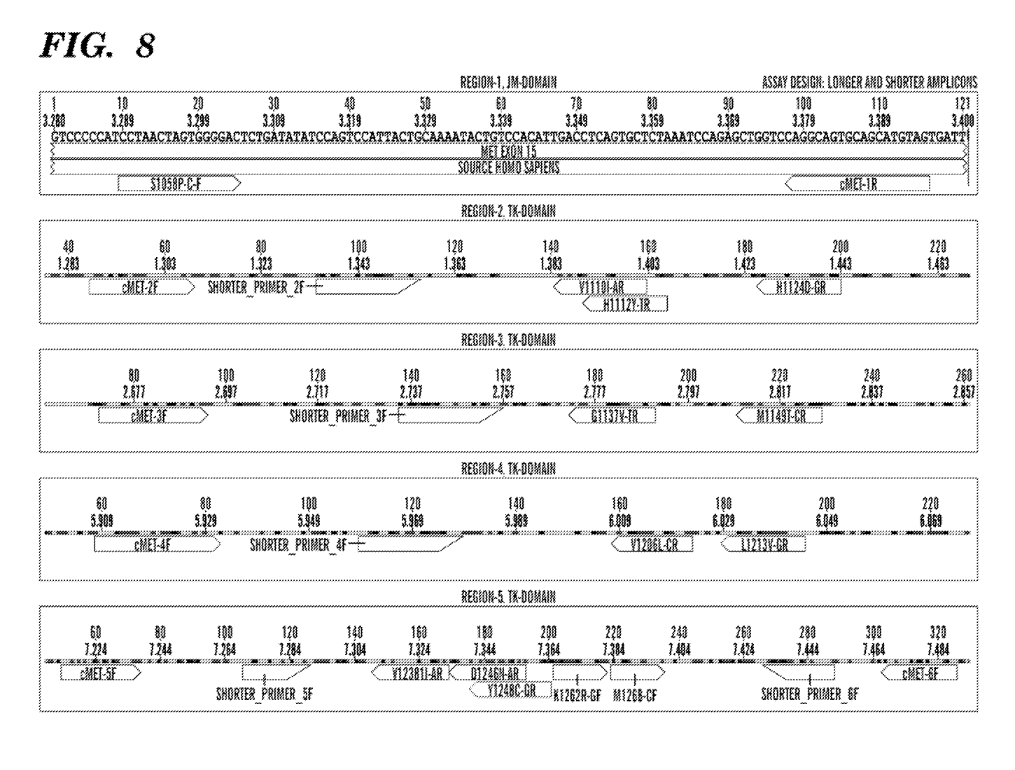

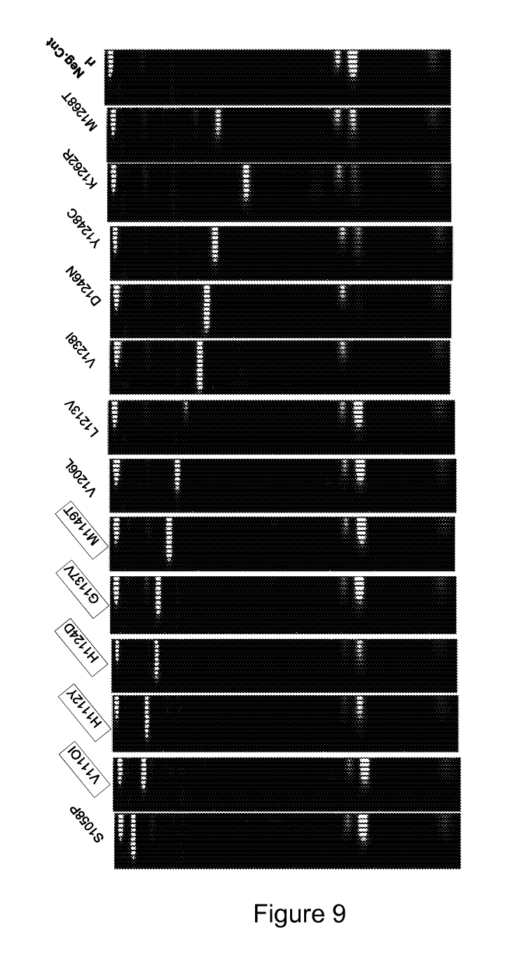

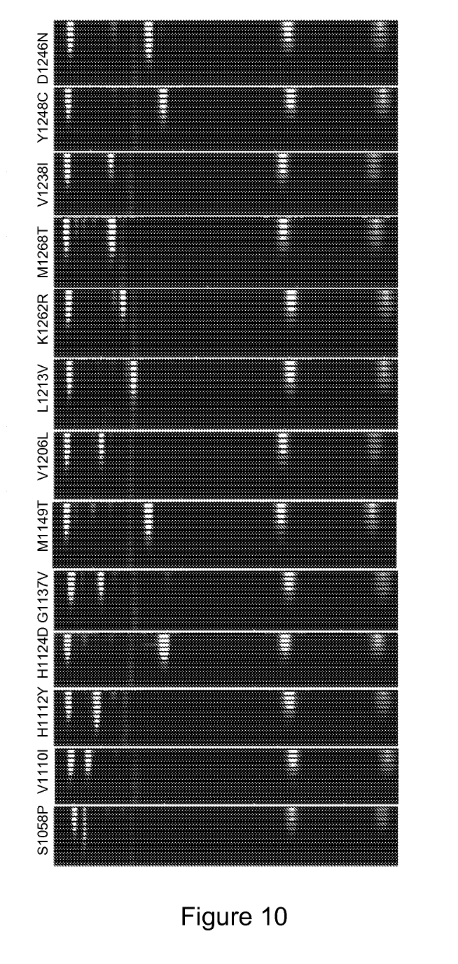

Detection of cMET snips was performed using the buffer, enzyme, and thermocycling parameters of Example 1. Two alternate sets of primers (FIG. 8), one amplifying longer amplicons (Table 3) and one amplifying shorter amplicons (Table 4) were tested, as shown in FIGS. 9-10.

### Example 3

### Relative Quantification of cMET and EGFR Copy Number Variation and cMET Gene Expression

Relative quantification of cMET and EGFR copy number variation and cMET gene expression was calculated according to Livak and Schmittgen, 2001, using a delta-delta Ct method. The assay was optimized to obtain similar PCR efficiencies for different targets ranging from 90-110%, and relative quantification for copy number variation and target expression was performed as described below:
Step 1: Calculate average Ct of cMET or EGFR CNV targets or cMET gene expression targets
Step 2: Calculate average Ct of reference genes. Two genes are used for copy number variation calculation, and two genes with two amplicons each were used to measure cMET gene expression.
Step 3: Calculate relative quantification by using the following formulae:

Fold difference relative to reference for cMET or EGFR CNV or cMET gene expression was calculated using the following formula:

=2^{(average Ct of cMET or EGFR or cMET gene expression-average Ct of reference genes)}

TABLE 3
Primers for detection of cMET SNPs - longer amplicons

| | | Core | Product | SEO ID |
|---|---|---|---|---|
| Mutation | Primer | Product | Length | NO |
| Region 1 | — | — | — | |
| S1058P-CF | aagggcaCCTAACTAGTGGGGACC | 107 | 107 + 7 + 6 = | 46 |
| | | | 120 bp | |
| cMET-1R | actcatCTACATGCTGCACTGCCTG | | | 47 |
| Region 2 | | | | |
| cMET-2F | ctccGAAGCTCATAAAGGGTTTGAT | | | 48 |
| V1110I-AR | cccggAACAAAGTCCCATGATATAT | 115 | 115 + 5 + 4 = | 49 |
| | | | 124 bp | |
| H1112Y-TR | ctgccGTCCAACAAAGTCCCATA | 119 | 119 + 5 + 4 = | 50 |
| | | | 128 bp | |
| H1124D-GR | taatacataacagtttGGATTTCACAGCACAGTC | 155 | 155 + 16 + 4 = | 51 |
| | | | 175bp | |
| Region 3 | | | | |
| cMET-3F | ccattCATTTCATTGCTCTTCCTATCTA | | | 52 |
| G1137V-TR | acaaccgAGAAATTGGGAAACTTCTA | 120 | 120 + 7 + 5 = | 53 |
| | | | 132 bp | |
| M1149T-CR | cacagcGGATGACTAAAATCTTTCG | 156 | 156 + 6 + 5 = | 54 |
| | | | 167 bp | |
| Region 4 | | | | |
| cMET-4F | caaattcaaaatAGGTCAAAATTAGAACAGTAG | | | 55 |
| | ATG | | | |
| V1206L-TR | accttctcaTCATGCCTTTGGCTAA | 115 | 115 + 9 + 12 = | 56 |
| | | | 136 bp | |
| L1213V-GR3 | ccccgAAACTTTTTGCTTGCtACA | 138 | 138 + 5 + 12 = | 57 |
| | | | 155 bp | |
| Region 5 | | | | |
| cMET-5F | cttcatataaattatTGTAGATATTCAGCATCATT | | | 58 |
| | GTAA | | | |
| V1238I-AR | acaaaacaaaatAAGACCAAAATCAGCAAT | 113 | 113 + 12 + 15 = | 59 |
| | | | 140 bp | |
| D1246N-AR | cgggcATAGTATTCTTTATCATACATGTT | 143 | 143 + 5 + 15 = | 60 |
| | | | 163 bp | |
| Y1248C-GR | cccccTGTACACTATAGTATTCTTTATCAC | 151 | 151 + 5 + 15 = | 61 |
| | | | 171 bp | |
| Region 6 | | | | |
| K1262R-GF | acactccataAACAAAACAGGTGCAAG | 124 | 124 + 10 + 14 = | 62 |
| | | | 148 bp | |
| M1268T-CF | ctttattattctatttactatttaCTGCCAGTGAAGTGGAC | 106 | 106 + 24 + 14 = | 63 |
| | | | 144 bp | |
| cMET-6R | ctcaaatatataatAAGTAAAAGAGGAGAAACTC | | | 64 |
| | AGA | | | |

TABLE 4
Primers for detection of cMET SNPs - shorter amplicons

| cMET | | | | Actual | |
|---|---|---|---|---|---|
| SNP | | | Core | size on | SEQ ID |
| Region | Primer Name | Primer Sequence | Size | ICEPlexer | NO |
| Region 1 | Genomic_Modified_S1058P- | TAGGATGGCCCCTAACTAGTGGGG | 107 | 117 | 65 |
| | CF | ACC | | | |
| | cMET-1R | /56- | | | 66 |
| | | FAM/ttaCTACATGCTGCACTGCCTG | | | |
| Region 2 | cMET-2F | /56- | | | 67 |
| | | FAM/AGAAGGACCGAAATTTTAAA | | | |
| | | ACGCAGTGCTAACCAAGTTCT | | | |
| | Genomic_Modified_V1110I- | AAGCTTCGTGATAAAATTAATTAA | 68 | 125 | 68 |
| | AR | TAATATATAATATTTTAACAAAGT | | | |
| | | CCCATGATATAT | | | |
| | Genomic_Modified_H1112Y- | ACCATGGTTTATAAAATTAATTAA | 72 | 129 | 69 |
| | TR | TAATATATAATATTTTGTCCAACA | | | |
| | | AAGTCCCATA | | | |
| | Genomic_Modified_H1124D- | ATCGGACTTCGGATTTCACAGCAC | 108 | 135 | 70 |
| | GR | AGTC | | | |
| Region 3 | cMET-3F | /56- | | | 71 |
| | | FAM/ATCGGACTTCTATTTTAATAA | | | |
| | | AATAATTTTATAATTAACTCCACC | | | |
| | | ACTGGATTTCTCAGG | | | |
| | Genomic_Modified_G1137V- | AACTTCTGGGAATATTTTTATATTA | 55 | 140 | 72 |
| | TR | AAAATATTTAAAATATTAAATAAG | | | |
| | | AAATTGGGAAACTTCTA | | | |
| | Genomic_Modified_M1149T- | TGAGTTACCAAATAAAAGGATGAC | 91 | 146 | 73 |
| | CR | TAAAATCTTTCG | | | |
| Region 4 | cMET-4F | /56- | | | 74 |
| | | FAM/TGGCAGTAGGATAAAATTAA | | | |
| | | TTAATAATATATAATATTTTTGACT | | | |
| | | GCAGAATCCAACTGT | | | |
| | Genomic_Modified_V1206L- | AGGCCACCGTATATAATTTTTTTA | 64 | 152 | 75 |
| | TR | AAAAATATTAATATTTTTATTTAAT | | | |
| | | CATGCCTTTGGCTAG | | | |
| | Genomic_Modified_L1213V- | AACCATACGAATTAATTAAAATTT | 86 | 158 | 76 |
| | GR3 | TTATATTTAAACTTTTTGCTTGCtACA | | | |
| Region 5 | cMET-5F | /56- | | | 77 |
| | | FAM/TTCCGTAAACTAATTAATAAT | | | |
| | | AAAATAATTTAATTATTGTCCTTTC | | | |
| | | TGTAGGCTGGATGA | | | |
| | Genomic_Modified_V1238I- | AACCATACGAAATTTTTTAAAATT | 57 | 164 | 78 |
| | AR | TTATAAATAAATATTTAAAATTTA | | | |
| | | AATATTAATTTAAAATTTTAAAAA | | | |
| | | GACCAAAATCAGCAAT | | | |
| | Genomic_Modified_D1246N- | TTGAGATGGCAATTTTTTATTATAA | 87 | 170 | 79 |
| | AR | ATTTTAATTTTTTAATTAATTATAG | | | |
| | | TATTCTTTATCATACATGTT | | | |
| | Genomic_Modified_Y1248C- | AGGAGAAGTCTTTATTAAATTATA | 95 | 175 | 80 |
| | GR | TAATTTAATTTTAAATTTTTGTACA | | | |
| | | CTATAGTATTCTTTATCAC | | | |
| Region 6 | Genomic_Modified_K1262R- | TGTGGAGATTAATTTTTTAAAATTT | 86 | 185 | 81 |
| | GF | TATAAATAAATATTTAAAATTTAA | | | |
| | | ATATTAATTTAATTAATTAAAATTT | | | |
| | | TTATATAACAAAACAGGTGCAAG | | | |
| | Genomic_Modified_M1268T- | TGTGGAGATTAATTTTTTAAAATTT | 68 | 180 | 82 |
| | CF | TATAAATAAATATTTAAAATTTAA | | | |
| | | ATATTAATTTAAATAATAATATTA | | | |
| | | CTGCCAGTGAAGTGGAC | | | |
| | cMET-6R | /56- | | | 83 |
| | | FAM/AGGCCACCGTAAAAATTAAA | | | |
| | | AATTAATAAATATTAATAAACCAC | | | |
| | | ATCTGACTTGGTGGTA | | | |

TABLE 5
Sample Characteristics

| | | Tissue | | MET Copy | MET | Chromosme | |
|---|---|---|---|---|---|---|---|
| Sample | Source | Origin | Matrix | Number | Expression | 7 Polysomy | Reference* |
| A549 | Cell Line | Lung | Fresh | 2 | Low | Unknown | 3 |
| | | | Frozen | | | | |
| H1993 | Cell Line | Lung | Fresh | >10 | High | Unknown | 3 |
| | | | Frozen | | | | |
| Lung | Tissue | Lung | Fresh | Unknown | Unknown | Unknown | |
| | | | Frozen | | | | |
| SNU-1 | Cell Line | Gastric | Fresh | 2 | No | No | 1, 2 |
| | | | Frozen | | | | |
| SNU-5 | Cell Line | Gastric | Fresh | >10 | High | Yes | 1, 2 |
| | | | Frozen | | | | |
| Gastric | Tissue | Stomach | Fresh | Unknown | Unknown | Unknown | |
| | | | Frozen | | | | |
| Gastric | Tissue | Stomach - | FFPE | Unknown | Unknown | Unknown | |
| | | Normal | | | | | |
| Gastric | Tissue | Stomach - | FFPE | Unknown | Unknown | Unknown | |
| | | Cancer | | | | | |

*(1) Catenacci D, Cancer BioTher, 2011, 12(1): 9-46 (2) Smolen G, PNAS, 2006 103(7): 2316-2321 (3) Lutterbach B, Cancer Res, 2007, 67: 2081

TABLE 6
Primers

| | | SEQ ID |
|---|---|---|
| Target | | NO: |
| Reverse Primers | | |
| gM1 | ATA AGC AGT GGC AGA AAT TC | 89 |
| gM2 | GTT AAG AGG CAG AAG AGA AC | 90 |
| gM3 | CAG GAT ATG CCA TGA ACA G | 91 |
| gE2 | CTG CCT GCT ACT GTA TGA | 92 |
| gE3 | TGT TAA AAG CCT ATT GGA GC | 93 |
| gE1 | CAT GTT GTG TGT ACA GAG T | 94 |
| gKDEL | TGG ACA TTT ATG TGG TGT G | 95 |
| (‘KDEL’ | | |
| disclosed as | | |
| SEQ ID NO: | | |
| 130) | | |
| gSOD | T GCT GCC TTA CAC AAC T | 96 |
| gSPG | CA GAA AAG TCA TCA GTG AGG | 97 |
| mM1 | GTC TGT CAG AGG ATA CTG C | 98 |
| mM3 | TTG TCC CTC CTT CAA GG | 99 |
| mM2 | GCT GGG GTA TAA CAT TCA AG | 100 |
| mKDEL-2 | A AAA AGA TCC AGG TAA CGA G | 101 |
| (‘KDEL’ | | |
| disclosed as | | |
| SEQ ID NO: | | |
| 130) | | |
| mKDEL-1 | TTT CAG GTA GAT CAG GTA CA | 102 |
| (‘KDEL’ | | |
| disclosed as | | |
| SEQ ID NO: | | |
| 130) | | |
| mSOD-2 | AGA GGA TTA AAG TGA GGA CC | 103 |
| mSOD-1 | ACT TTC TTC ATT TCC ACC TT | 104 |
| mSPG-2 | G CCA GAT GAA AAA TTT CCA A | 105 |
| mSPG-1 | CAT GGA ATT GCA GCA AAT G | 106 |
| Forward Primers | | |
| gM1 | CTA TGT TCT TAT CTC CTC AGT | 107 |
| gM2 | G GTT CCA TCC TAG CTC TT | 108 |
| gM3 | AC TCA CCC ACT CTC TGA T | 109 |
| gE2 | AC CCA GTG ACT TAC CTA TG | 110 |
| gE3 | T TCA AAT CTG GAA AGG ACA C | 111 |
| gE1 | CT TCT GGG GAA GCT CAT T | 112 |
| gKDEL | CA GCA TCT GAA ACC CAT AG | 113 |
| (‘KDEL’ | | |
| disclosed as | | |
| SEQ ID NO: | | |
| 130) | | |
| gSOD | G TGC TCT GTG AAT GTC ATC | 114 |
| gSPG | TA CCC AGG TTT CCA GAA TAG | 115 |
| mM1 | C TGT AGA CTA CCG AGC TAC | 116 |
| mM3 | T CGG AGG AAT GCC TGA | 117 |
| mM2 | C TAA ACA GTG GGA ATT CTA | 118 |
| | GAC | |
| mKDEL-2 | CTG CTG AAG ATC TGG AAG A | 119 |
| (‘KDEL’ | | |
| disclosed as | | |
| SEQ ID NO: | | |
| 130) | | |
| mKDEL-1 | CTT CAT TTA TTT CAT TGT ATA | 120 |
| (‘KDEL’ | ACA CA | |
| disclosed as | | |
| SEQ ID NO: | | |
| 130) | | |
| mSOD-2 | G CAT TAA AGG ACT GAC TGA A | 121 |
| mSOD-1 | GAT CTC ACT CTC AGG AGA | 122 |
| mSPG-2 | AC ACC TCT ATC TTC AAC CAA | 123 |
| mSPG-1 | G CTC ATC TGA AAA CAG GAG | 124 |

1.-26. (canceled)

27. A method of detecting cMET alterations, the method comprising
contacting a portion of a nucleic acid sample with a set of primers which detect alterations in cMET gene copy number variation;
wherein the set of primers comprises subsets of primer pairs that amplify at least one gDNA-specific sequence of cMET and at least one gDNA-specific sequence of each of at least two reference genes, wherein one reference gene is located on chromosome 7 and one reference gene is not located on chromosome 7 to detect cMET gene copy number variation;
performing a PCR amplification regimen comprising cycles of strand separation, primer annealing, and primer extension on a reaction mixture comprising the portion of the sample and the set of primers;
detecting the level of the amplicon for each primer pair;
normalizing the level of cMET amplicons to the reference gene amplicons;
and comparing the normalized level of cMET amplicons to a reference level; wherein a higher level of a gDNA-specific cMET amplicon as compared to the reference level indicates the presence of a gene amplification alteration of cMET in the sample.

28. The method of claim 27, wherein the set of primers further comprises a subset of primer pairs that amplify at least one gDNA-specific sequence of EGFR; and
the assay further comprises comparing the normalized level of EGFR amplicons to a reference level; wherein a higher level of a gDNA-specific EGFR amplicon as compared to the reference level indicates the presence of a gene amplification alteration of EGFR in the sample.

29. The method of claim 27, wherein the reference gene of the primer set which is located on chromosome 7 is KDELR-2; and
the method further comprises comparing the normalized level of KDELR-2 amplicons to a reference level; wherein a higher level of a gDNA-specific KDELR-2 amplicon as compared to the reference level indicates the presence of a gene amplification alteration of KDELR-2 in the sample.

30. The method of claim 27, wherein the presence of a gene amplification alteration of cMET, EGFR and KDELR-2 indicates the presence of chromosome 7 amplification.

31. The method of claim 27, wherein the reference gene of the primer set which is not located on chromosome 7 is SOD1 or SPG21.

32. The method of claim 31, wherein the primer set comprises subsets of primer pairs that amplify at least one gDNA-specific sequence of SOD1 and SPG21.

33. The method of claim 27, further comprising
contacting the portion of a nucleic acid sample with a second set of primers, wherein the second set of primers detects changes in cMET gene expression level;
wherein the second set of primers comprises subsets of primer pairs that amplify mRNA-specific sequences of cMET and at least mRNA specific sequences of at least two reference genes; and
wherein an altered level of a mRNA-specific cMET amplicon as compared to the reference level indicates the presence of a gene expression level alteration of cMET in the sample.

34. The method of claim 27, wherein the first primer set comprises subsets of primer pairs that amplify at least one gDNA-specific sequence of each of SOD1 and SPG21.

35. The method of claim 27, wherein a primer set comprises primer pair subsets that amplify at least one amplicon of each gene.

36. (canceled)

37. (canceled)

38. The method of claim 27, wherein the primer sets comprise primer pair subsets that amplify at least two gDNA-specific amplicons of each of cMET, EGFR, and KDELR-2 and at least two mRNA-specific amplicons of each of cMET, SOD1 and SGP21.

39. (canceled)

40. The method of claim 27, further comprising:
contacting a second portion of the sample with a third set of primer pairs wherein the third set of primers comprises subsets of primer pairs that amplify cMET sequences comprising sequence variations;
performing a PCR amplification regimen comprising cycles of strand separation, primer annealing, and primer extension on a reaction mixture comprising the second portion of the sample and the third set of primers;
detecting the level of the amplicon for each primer pair, wherein the presence of an amplicon indicates the presence of the sequence variation for which that primer pair is specific.

41. The method of claim 40, wherein one or more sequence variations of cMET are SNPs.

42. The method of claim 41, wherein the cMET SNP is selected from the group consisting of:
S1058P; V1101I; H1112Y; H1124D; G1137V; M1149T; V1206L; L1213V; K1262R; M1268T; V12381; Y1248C; and D1246N (SEQ ID NO: 131).

43. (canceled)

44. The method of claim 40, wherein the same PCR thermocycling regimens are used for both reactions.

45. The method of claim 27, wherein the nucleic acid sample is prepared from a FFPE tumor sample.

46. The method of claim 27, wherein the sample comprises tumor cells from a subject diagnosed with a condition selected from the group consisting of:
gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.

47. The method of claim 27, wherein one or more primers are dual domain primers.

48. The method of claim 27, wherein an amplified products from two or more primer pairs of a primer subset can be distinguished.

49. The method of claim 27, wherein the amplified products from two or more primer pairs of a primer subset are distinguished by being of distinct sizes or by being labeled with different detectable labels.

50. (canceled)

51. The method of claim 27, wherein the amplified products from the first set of primers and the second set of primers are distinguished by being labeled with different detectable labels.

52. The method of claim 27, wherein one or more primers are selected from the group consisting of SEQ ID NOs: 1-83.

53. The method of claim 27, wherein one or more primers comprise a sequence of any of SEQ ID NOs: 89-124.

54. (canceled)


SEQUENCE LISTING (132 sequences)

SEQ ID NO: 1 - DNA, 17 bp, Artificial Sequence
  source: /note=“Description of Artificial Sequence Synthetic primer”
gtctgtcagaggatact

SEQ ID NO: 2 - DNA, 16 bp, Artificial Sequence
  source: /note=“Description of Artificial Sequence Synthetic primer”
ttgtccctccttcaag

SEQ ID NO: 3 - DNA, 17 bp, Artificial Sequence
  source: /note=“Description of Artificial Sequence Synthetic primer”
gctggggtataacattc

SEQ ID NO: 4 - DNA, 18 bp, Artificial Sequence
  source: /note=“Description of Artificial Sequence Synthetic primer”
aaaaagatccaggtaacg

SEQ ID NO: 5 - DNA, 17 bp, Artificial Sequence
  source: /note=“Description of Artificial Sequence Synthetic primer”
tttcaggtagatcaggt

SEQ ID NO: 6 - DNA, 18 bp, Artificial Sequence
  source: /note=“Description of Artificial Sequence Synthetic primer”
agaggattaaagtgagga

SEQ ID NO: 7 - DNA, 18 bp, Artificial Sequence
  source: /note=“Description of Artificial Sequence Synthetic primer”
actttcttcatttccacc

SEQ ID NO: 8 - DNA, 18 bp, Artificial Sequence
  source: /note=“Description of Artificial Sequence Synthetic primer”
gccagatgaaaaatttcc

SEQ ID NO: 9 - DNA, 16 bp, Artificial Sequence
  source: /note=“Description of Artificial Sequence Synthetic primer”
catggaattgcagcaa

SEQ ID NO: 10 - DNA, 42 bp, Artificial Sequence
  source: /note=“Description of Artificial Sequence Synthetic primer”
aacgcccgctttattaatattctatgttcttatctcctcagt

SEQ ID NO: 11 - DNA, 47 bp, Artificial Sequence
  source: /note=“Description of Artificial Sequence Synthetic primer”
tgagttaccattaaaataataaattaattggttccatcctagctctt

SEQ ID NO: 12 - DNA, 49 bp, Artificial Sequence
  source: /note=“Description of Artificial Sequence Synthetic primer”
agcttaaacgaaatattaaatattattattaactcacccactctctgat

SEQ ID NO: 13 - DNA, 38 bp, Artificial Sequence
  source: /note=“Description of Artificial Sequence Synthetic primer”
tcagaaggacaatatttttacccagtgacttacctatg

SEQ ID NO: 14 - DNA, 58 bp, Artificial Sequence
  source: /note=“Description of Artificial Sequence Synthetic primer”
tatcgtaacataatttaataataaaataatttaattatttcaaatctggaaaggacac

SEQ ID NO: 15 - DNA, 34 bp, Artificial Sequence
  source: /note=“Description of Artificial Sequence Synthetic primer”
tcctgcgctgtataaacttctggggaagctcatt

SEQ ID NO: 16 - DNA, 53 bp, Artificial Sequence
  source: /note=“Description of Artificial Sequence Synthetic primer”
actttgcctaaataaatattaataattaatatatcagcatctgaaacccatag

SEQ ID NO: 17 - DNA, 60 bp, Artificial Sequence
  source: /note=“Description of Artificial Sequence Synthetic primer”
taaactccctataaaattaaattaataatatataatattttgtgctctgtgaatgtcatc

SEQ ID NO: 18 - DNA, 60 bp, Artificial Sequence
  source: /note=“Description of Artificial Sequence Synthetic primer”
tgtggagattataaaattaattaataatatataatatttttacccaggtttccagaatag

SEQ ID NO: 19 - DNA, 39 bp, Artificial Sequence
  source: /note=“Description of Artificial Sequence Synthetic primer”
aagcttcgtgataattaaatctgtagactaccgagctac

SEQ ID NO: 20 - DNA, 54 bp, Artificial Sequence
  source: /note=“Description of Artificial Sequence Synthetic primer”
taggatggcctattttaataaaataattttataattaatcggaggaatgcctga

SEQ ID NO: 21 - DNA, 51 bp, Artificial Sequence
  source: /note=“Description of Artificial Sequence Synthetic primer”
agaaggaccgttttatttattttattatactaaacagtgggaattctagac

SEQ ID NO: 22 - DNA, 55 bp, Artificial Sequence
  source: /note=“Description of Artificial Sequence Synthetic primer”
ttgagatggcattaattaaatttttaataatatttactgctgaagatctggaaga

SEQ ID NO: 23 - DNA, 47 bp, Artificial Sequence
  source: /note=“Description of Artificial Sequence Synthetic primer”
actttgcctaaatatatttttcttcatttatttcattgtataacaca

SEQ ID NO: 24 - DNA, 67 bp, Artificial Sequence
  source: /note=“Description of Artificial Sequence Synthetic primer”
atctatataaataattttataaaataatttattaaaattaaatatatgcattaaaggactgactgaa

SEQ ID NO: 25 - DNA, 42 bp, Artificial Sequence
  source: /note=“Description of Artificial Sequence Synthetic primer”
accatggtttataataaatattaagatctcactctcaggaga

SEQ ID NO: 26 - DNA, 75 bp, Artificial Sequence
  source: /note=“Description of Artificial Sequence Synthetic primer”
aagcagcagataatttattatataattaaaaataattataattaataaaatttaaacacctctatcttcaaccaa

SEQ ID NO: 27 - DNA, 57 bp, Artificial Sequence
  source: /note=“Description of Artificial Sequence Synthetic primer”
accatctcggtaattaataattaaaataatttaattatgctcatctgaaaacaggag

SEQ ID NO: 28 - DNA, 35 bp, Artificial Sequence
  source: /note=“Description of Artificial Sequence Synthetic primer”
tcattgcccttttaaataagcagtggcagaaattc

SEQ ID NO: 29 - DNA, 35 bp, Artificial Sequence
  source: /note=“Description of Artificial Sequence Synthetic primer”
agcatgcgtatttaagttaagaggcagaagagaac

SEQ ID NO: 30 - DNA, 34 bp, Artificial Sequence
  source: /note=“Description of Artificial Sequence Synthetic primer”
atagctgttatttaacaggatatgccatgaacag

SEQ ID NO: 31 - DNA, 33 bp, Artificial Sequence
  source: /note=“Description of Artificial Sequence Synthetic primer”
atgatggagttttaactgcctgctactgtatga

SEQ ID NO: 32 - DNA, 35 bp, Artificial Sequence
  source: /note=“Description of Artificial Sequence Synthetic primer”
aggccaccgttttaatgttaaaagcctattggagc

SEQ ID NO: 33 - DNA, 34 bp, Artificial Sequence
  source: /note=“Description of Artificial Sequence Synthetic primer”
ttcatgcaattttaacatgttgtgtgtacagagt

SEQ ID NO: 34 - DNA, 46 bp, Artificial Sequence
  source: /note=“Description of Artificial Sequence Synthetic primer”
aggagaagtctttttatatttattatatggacatttatgtggtgtg

SEQ ID NO: 35 - DNA, 49 bp, Artificial Sequence
  source: /note=“Description of Artificial Sequence Synthetic primer”
actagttgctattaattaaaatttttatattttgctgccttacacaact

SEQ ID NO: 36 - DNA, 54 bp, Artificial Sequence
  source: /note=“Description of Artificial Sequence Synthetic primer”
actagttgctatttaataataaatttaaaaatatcagaaaagtcatcagtgagg

SEQ ID NO: 37 - DNA, 34 bp, Artificial Sequence
  source: /note=“Description of Artificial Sequence Synthetic primer”
ttgcgatccctttaagtctgtcagaggatactgc

SEQ ID NO: 38 - DNA, 32 bp, Artificial Sequence
  source: /note=“Description of Artificial Sequence Synthetic primer”
aaacttcgcatttaattgtccctccttcaagg

SEQ ID NO: 39 - DNA, 35 bp, Artificial Sequence
  source: /note=“Description of Artificial Sequence Synthetic primer”
tcgcgctagatttaagctggggtataacattcaag

SEQ ID NO: 40 - DNA, 46 bp, Artificial Sequence
  source: /note=“Description of Artificial Sequence Synthetic primer”
tttatgccatttataataatataaaaaaaaagatccaggtaacgag

SEQ ID NO: 41 - DNA, 35 bp, Artificial Sequence
  source: /note=“Description of Artificial Sequence Synthetic primer”
aggagaagtctttaatttcaggtagatcaggtaca

SEQ ID NO: 42 - DNA, 35 bp, Artificial Sequence
  source: /note=“Description of Artificial Sequence Synthetic primer”
ttccgtaaactttaaagaggattaaagtgaggacc

SEQ ID NO: 43 - DNA, 35 bp, Artificial Sequence
  source: /note=“Description of Artificial Sequence Synthetic primer”
aaccatacgatttaaactttcttcatttccacctt

SEQ ID NO: 44 - DNA, 37 bp, Artificial Sequence
  source: /note=“Description of Artificial Sequence Synthetic primer”
tgcataagaatttaatagccagatgaaaaatttccaa

SEQ ID NO: 45 - DNA, 34 bp, Artificial Sequence
  source: /note=“Description of Artificial Sequence Synthetic primer”
aggagaagtctttaacatggaattgcagcaaatg

SEQ ID NO: 46 - DNA, 24 bp, Artificial Sequence
  source: /note=“Description of Artificial Sequence Synthetic primer”
aagggcacctaactagtggggacc

SEQ ID NO: 47 - DNA, 25 bp, Artificial Sequence
  source: /note=“Description of Artificial Sequence Synthetic primer”
actcatctacatgctgcactgcctg

SEQ ID NO: 48 - DNA, 25 bp, Artificial Sequence
  source: /note=“Description of Artificial Sequence Synthetic primer”
ctccgaagctcataaagggtttgat

SEQ ID NO: 49 - DNA, 25 bp, Artificial Sequence
  source: /note=“Description of Artificial Sequence Synthetic primer”
cccggaacaaagtcccatgatatat

SEQ ID NO: 50 - DNA, 23 bp, Artificial Sequence
  source: /note=“Description of Artificial Sequence Synthetic primer”
ctgccgtccaacaaagtcccata

SEQ ID NO: 51 - DNA, 34 bp, Artificial Sequence
  source: /note=“Description of Artificial Sequence Synthetic primer”
taatacataacagtttggatttcacagcacagtc

SEQ ID NO: 52 - DNA, 28 bp, Artificial Sequence
  source: /note=“Description of Artificial Sequence Synthetic primer”
ccattcatttcattgctcttcctatcta

SEQ ID NO: 53 - DNA, 26 bp, Artificial Sequence
  source: /note=“Description of Artificial Sequence Synthetic primer”
acaaccgagaaattgggaaacttcta

SEQ ID NO: 54 - DNA, 25 bp, Artificial Sequence
  source: /note=“Description of Artificial Sequence Synthetic primer”
cacagcggatgactaaaatctttcg

SEQ ID NO: 55 - DNA, 36 bp, Artificial Sequence
  source: /note=“Description of Artificial Sequence Synthetic primer”
caaattcaaaataggtcaaaattagaacagtagatg

SEQ ID NO: 56 - DNA, 25 bp, Artificial Sequence
  source: /note=“Description of Artificial Sequence Synthetic primer”
accttctcatcatgcctttggctaa

SEQ ID NO: 57 - DNA, 24 bp, Artificial Sequence
  source: /note=“Description of Artificial Sequence Synthetic primer”
ccccgaaactttttgcttgctaca

SEQ ID NO: 58 - DNA, 39 bp, Artificial Sequence
  source: /note=“Description of Artificial Sequence Synthetic primer”
cttcatataaattattgtagatattcagcatcattgtaa

SEQ ID NO: 59 - DNA, 30 bp, Artificial Sequence
  source: /note=“Description of Artificial Sequence Synthetic primer”
acaaaacaaaataagaccaaaatcagcaat

SEQ ID NO: 60 - DNA, 29 bp, Artificial Sequence
  source: /note=“Description of Artificial Sequence Synthetic primer”
cgggcatagtattctttatcatacatgtt

SEQ ID NO: 61 - DNA, 30 bp, Artificial Sequence
  source: /note=“Description of Artificial Sequence Synthetic primer”
ccccctgtacactatagtattctttatcac

SEQ ID NO: 62 - DNA, 27 bp, Artificial Sequence
  source: /note=“Description of Artificial Sequence Synthetic primer”
acactccataaacaaaacaggtgcaag

SEQ ID NO: 63 - DNA, 41 bp, Artificial Sequence
  source: /note=“Description of Artificial Sequence Synthetic primer”
ctttattattctatttactatttactgccagtgaagtggac

SEQ ID NO: 64 - DNA, 37 bp, Artificial Sequence
  source: /note=“Description of Artificial Sequence Synthetic primer”
ctcaaatatataataagtaaaagaggagaaactcaga

SEQ ID NO: 65 - DNA, 27 bp, Artificial Sequence
  source: /note=“Description of Artificial Sequence Synthetic primer”
taggatggcccctaactagtggggacc

SEQ ID NO: 66 - DNA, 22 bp, Artificial Sequence
  source: /note=“Description of Artificial Sequence Synthetic primer”
ttactacatgctgcactgcctg

SEQ ID NO: 67 - DNA, 41 bp, Artificial Sequence
  source: /note=“Description of Artificial Sequence Synthetic primer”
agaaggaccgaaattttaaaacgcagtgctaaccaagttct

SEQ ID NO: 68 - DNA, 60 bp, Artificial Sequence
  source: /note=“Description of Artificial Sequence Synthetic primer”
aagcttcgtgataaaattaattaataatatataatattttaacaaagtcccatgatatat

SEQ ID NO: 69 - DNA, 58 bp, Artificial Sequence
  source: /note=“Description of Artificial Sequence Synthetic primer”
accatggtttataaaattaattaataatatataatattttgtccaacaaagtcccata

SEQ ID NO: 70 - DNA, 28 bp, Artificial Sequence
  source: /note=“Description of Artificial Sequence Synthetic primer”
atcggacttcggatttcacagcacagtc

SEQ ID NO: 71 - DNA, 60 bp, Artificial Sequence
  source: /note=“Description of Artificial Sequence Synthetic primer”
atcggacttctattttaataaaataattttataattaactccaccactggatttctcagg

SEQ ID NO: 72 - DNA, 66 bp, Artificial Sequence
  source: /note=“Description of Artificial Sequence Synthetic primer”
aacttctgggaatatttttatattaaaaatatttaaaatattaaataagaaattgggaaacttcta

SEQ ID NO: 73 - DNA, 36 bp, Artificial Sequence
  source: /note=“Description of Artificial Sequence Synthetic primer”
tgagttaccaaataaaaggatgactaaaatctttcg

SEQ ID NO: 74 - DNA, 60 bp, Artificial Sequence
  source: /note=“Description of Artificial Sequence Synthetic primer”
tggcagtaggataaaattaattaataatatataatatttttgactgcagaatccaactgt

SEQ ID NO: 75 - DNA, 64 bp, Artificial Sequence
  source: /note=“Description of Artificial Sequence Synthetic primer”
aggccaccgtatataatttttttaaaaaatattaatatttttatttaatcatgcctttggctag

SEQ ID NO: 76 - DNA, 51 bp, Artificial Sequence
  source: /note=“Description of Artificial Sequence Synthetic primer”
aaccatacgaattaattaaaatttttatatttaaactttttgcttgctaca

SEQ ID NO: 77 - DNA, 60 bp, Artificial Sequence
  source: /note=“Description of Artificial Sequence Synthetic primer”
ttccgtaaactaattaataataaaataatttaattattgtcctttctgtaggctggatga

SEQ ID NO: 78 - DNA, 88 bp, Artificial Sequence
  source: /note=“Description of Artificial Sequence Synthetic primer”
aaccatacgaaattttttaaaattttataaataaatatttaaaatttaaatattaatttaaaattttaaaaagaccaaaatcagcaat

SEQ ID NO: 79 - DNA, 70 bp, Artificial Sequence
  source: /note=“Description of Artificial Sequence Synthetic primer”
ttgagatggcaattttttattataaattttaattttttaattaattatagtattctttatcatacatgtt

SEQ ID NO: 80 - DNA, 68 bp, Artificial Sequence
  source: /note=“Description of Artificial Sequence Synthetic primer”
aggagaagtctttattaaattatataatttaattttaaatttttgtacactatagtattctttatcac

SEQ ID NO: 81 - DNA, 97 bp, Artificial Sequence
  source: /note=“Description of Artificial Sequence Synthetic primer”
tgtggagattaattttttaaaattttataaataaatatttaaaatttaaatattaatttaattaattaaaatttttatataacaaaacaggtgcaag

SEQ ID NO: 82 - DNA, 90 bp, Artificial Sequence
  source: /note=“Description of Artificial Sequence Synthetic primer”
tgtggagattaattttttaaaattttataaataaatatttaaaatttaaatattaatttaaataataatattactgccagtgaagtggac

SEQ ID NO: 83 - DNA, 60 bp, Artificial Sequence
  source: /note=“Description of Artificial Sequence Synthetic primer”
aggccaccgtaaaaattaaaaattaataaatattaataaaccacatctgacttggtggta

SEQ ID NO: 84 - DNA, 6695 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 85 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 86 - DNA, 2874 bp, Homo sapiens
agggtgccccgcgcgcgcgcgcgccggcagttcggccacgtccctggccacgtcgcgggcgatctcgccatcttcgccgcttcctctcaggggccgccgcctcctgagccgcccagccccggggccgccgcgctgcgccgaccgccaccgccgccgccgccatgaacattttccggctgactggggacctgtcccacctggcggccatcgtcatcctgctgctgaagatctggaagacgcgctcctgcgccggtatttctgggaaaagccagcttctgtttgcactggtcttcacaactcgttacctggatctttttacttcatttatttcattgtataacacatctatgaaggttatctaccttgcctgctcctatgccacagtgtacctgatctacctgaaatttaaggcaacctacgatggaaatcatgataccttccgagtggagtttctggtggtccctgtgggaggcctctcatttttagttaatcacgatttctctcctcttgagatcctctggaccttctccatctacctggagtccgtggctatccttccgcagctatttatgatcagcaagactggggaggccgagaccatcaccacccactacctgttcttcctgggcctctatcgtgctttgtatcttgtcaactggatctggcgcttctactttgagggcttctttgacctcattgctgtggtggccggcgtagtccagaccatcctatactgtgacttcttctacttgtacattacaaaagtactcaagggaaagaagctcagtttgccagcataagtgccaaagaccatcaccagcatctgtccttcagggtgctcggacagaattcttaccacagcaaaggcataagatgcttgatacggaaaatcagaaacttaactcttttgttgcagatagtcatcagtggctctgtaaaaacgcagaggaaaagagccagaaggtttctgtttaatgcatcttgccttatctttttttattactgtgtacaaagatttttttacacaaagaaacttaatgctgtattaataaattcagtgtgtagcttcaattgggatagttccaaaagtgaagattttgtgaggaataagtgcaaattttttttttattttaaaaaattctttgaaactcttaagtctttgtgtctgcaatgaaattgtactccttgacagttgatagattatatattcttccatccctcaaacttgcattccactatatttattttttggcaaaagatgagctgtatttgtttgaaatctgagacactatgttcaattggatgtatctgttcaaatttattcccacgtgacgtggaagtccttcgttggatgtcacaacactacatttaaggttggtaaggatgacttggaggtccatggttttcattaccaacattttaagattctgaatgtcgatggagtctcactgaagagtcaccaaaggtgcctgccctcctcccctgctgggaagtgtcagttggagactgtcccaagggtgctgaagaatccagtggcaggggttctggctgctttccatctgagtgtgggatgggaggggtgttcatgatcatttggatatagcaatctactctgagaaatggaacacaaggagttacctatcactttcacttataattccaaaagatgactacaaccatgtccatgctcagattcaaacagttttccatatcacttttgggtggtaagatgatttaattacagttttttttttaattggcagcaccactaaccattccttacattcttttttgtatgtgtggttttctttttatttaacccgcagccgacatcgtagtttcttgttttgttttgttttacagagctgttgcatgacttatgttaccatcctaaaaaacactatattaaacatggaataaattgtctttttatgaattaggctttttgaacatcctgtgttgggatttttttgtttttcaattggcaacaaaagctctgtagggctgcagacatttaaagttcacataatcatctgtaagacattatgtattttgtggaaatactagaatttttttcctgattttgccattatatggatttgctattttttgattaatgcaaaagtatatgactttgttttttatgtgatacaccataaatattaaagtgttgaatactaacagtgctgactacaagaaggatgtagtattttggctttctgcattacaacgtcttctggaggaagggagcaggatgggtttcatttgtatctagtgtgtgtcttaacatctttctaaatcggcagtgtaactgcatagtttaacttcctgtctgtctccctcattttactcttccctccttgtcttatgttttggcttttgtgtatcagagcatctttctttgcagcatcctagtcttgcttcagtttctgtggtctcttttcatcctgcttgaagattcggagtgtggaaggaggctaggaagtctggtgcagtaggtgagcagacctcttgctgcccagcccagggtgggtggggcgcacacctgtctttgtgcatgcaaatctgatacacctggcgcatcctctggagagcacaacgcatggaaaggtctggaagctctgtgtagccattccttctgcagtcatcctacccaagtaaaagtaaccttggctatgttaccaccgttttggtcacccaggaggacatcttagcaagggtgcctgcgagggagtgtgggactgggcctcatcctcgccggcgttggaaaccaaggccttgtatgccacgccttatgaagcactgtttcacagttactttcacttcccgaataaaggttaccaggtaattaatatttttaaaaaaaaaaaaaaa

SEQ ID NO: 87 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 88 - DNA, 1635 bp, Homo sapiens
gggtggtcggtgtgcttgtgaccctgcctttgtgtggctgtcaccggtgggactggcggggagctgtgtgattaacctccatttcagctaatcatgggagagattaaagtctctcctgattataactggtttagaggtacagttccccttaaaaagattattgtggatgatgatgacagtaagatatggtcgctctatgacgcgggcccccgaagtatcaggtgtcctctcatattcctgccccctgtcagtggaactgcagatgtctttttccggcagattttggctctgactggatggggttaccgggttatcgctttgcagtatccagtttattgggaccatctcgagttctgtgatggattcagaaaacttttagaccatttacaattggataaagttcatctttttggcgcttctttgggaggctttttggcccagaaatttgctgaatacactcacaaatctcctagagtccattccctaatcctctgcaattccttcagtgacacctctatcttcaaccaaacttggactgcaaacagcttttggctgatgcctgcatttatgctcaaaaaaatagttcttggaaatttttcatctggcccggtggaccctatgatggctgatgccattgatttcatggtagacaggctagaaagtttgggtcagagtgaactggcttcaagacttaccttgaattgtcaaaattcttatgtggaacctcataaaattcgggacatacctgtaactattatggatgtgtttgatcagagtgcgctttcaactgaagctaaagaagaaatgtacaagctgtatcctaatgcccgaagagctcatctgaaaacaggaggcaatttcccatacctgtgcagaagtgcagaggtcaatctttatgtacagatacatttgctgcaattccatggaaccaaatacgcggccattgacccatcaatggtcagtgccgaggagcttgaggtgcagaaaggcagccttggcatcagccaggaggagcagtagtgtgtctctcgctgtcaatgatgagttgacccggtgtgttcttgtatagtcagtggcatcagcacccgtcagccggccttttccttcaggttcgtcaggctcaccggttctcactgtgtctgggaagtaggactgatggtcatcttcatgacaggcggcatctccactaagcctgtgtaactgttccctctttggttttcttagcttttgaatttgaagaagtacttttgaagactcccattttaagaaccgtgcagattttgctaccaaaagtcttcaccactgtgttcttaagtgaatgttaatttctgaggtttgggactttgtggtggtttttttcttcttttcttttccattcttctttctttctttttatgttgtttgctgtaaatgctgcacatccagattgcatatcaggacattggttattttatgctttcttggatataaccatgatcagagtgccatggccactaccccactgtttgctctcctgcaaatcaactgcttttaatttacacttaaacaaattgttttgagtgttagctactgcctttctagatattagtcatttggaataaaaattcaatttcactgaaaaaaaaaaaaaaaaaa

SEQ ID NO: 89 - DNA, 20 bp, Artificial Sequence
  source: /note=“Description of Artificial Sequence Synthetic primer”
ataagcagtggcagaaattc

SEQ ID NO: 90 - DNA, 20 bp, Artificial Sequence
  source: /note=“Description of Artificial Sequence Synthetic primer”
gttaagaggcagaagagaac

SEQ ID NO: 91 - DNA, 19 bp, Artificial Sequence
  source: /note=“Description of Artificial Sequence Synthetic primer”
caggatatgccatgaacag

SEQ ID NO: 92 - DNA, 18 bp, Artificial Sequence
  source: /note=“Description of Artificial Sequence Synthetic primer”
ctgcctgctactgtatga

SEQ ID NO: 93 - DNA, 20 bp, Artificial Sequence
  source: /note=“Description of Artificial Sequence Synthetic primer”
tgttaaaagcctattggagc

SEQ ID NO: 94 - DNA, 19 bp, Artificial Sequence
  source: /note=“Description of Artificial Sequence Synthetic primer”
catgttgtgtgtacagagt

SEQ ID NO: 95 - DNA, 19 bp, Artificial Sequence
  source: /note=“Description of Artificial Sequence Synthetic primer”
tggacatttatgtggtgtg

SEQ ID NO: 96 - DNA, 17 bp, Artificial Sequence
  source: /note=“Description of Artificial Sequence Synthetic primer”
tgctgccttacacaact

SEQ ID NO: 97 - DNA, 20 bp, Artificial Sequence
  source: /note=“Description of Artificial Sequence Synthetic primer”
cagaaaagtcatcagtgagg

SEQ ID NO: 98 - DNA, 19 bp, Artificial Sequence
  source: /note=“Description of Artificial Sequence Synthetic primer”
gtctgtcagaggatactgc

SEQ ID NO: 99 - DNA, 17 bp, Artificial Sequence
  source: /note=“Description of Artificial Sequence Synthetic primer”
ttgtccctccttcaagg

SEQ ID NO: 100 - DNA, 20 bp, Artificial Sequence
  source: /note=“Description of Artificial Sequence Synthetic primer”
gctggggtataacattcaag

SEQ ID NO: 101 - DNA, 20 bp, Artificial Sequence
  source: /note=“Description of Artificial Sequence Synthetic primer”
aaaaagatccaggtaacgag

SEQ ID NO: 102 - DNA, 20 bp, Artificial Sequence
  source: /note=“Description of Artificial Sequence Synthetic primer”
tttcaggtagatcaggtaca

SEQ ID NO: 103 - DNA, 20 bp, Artificial Sequence
  source: /note=“Description of Artificial Sequence Synthetic primer”
agaggattaaagtgaggacc

SEQ ID NO: 104 - DNA, 20 bp, Artificial Sequence
  source: /note=“Description of Artificial Sequence Synthetic primer”
actttcttcatttccacctt

SEQ ID NO: 105 - DNA, 20 bp, Artificial Sequence
  source: /note=“Description of Artificial Sequence Synthetic primer”
gccagatgaaaaatttccaa

SEQ ID NO: 106 - DNA, 19 bp, Artificial Sequence
  source: /note=“Description of Artificial Sequence Synthetic primer”
catggaattgcagcaaatg

SEQ ID NO: 107 - DNA, 21 bp, Artificial Sequence
  source: /note=“Description of Artificial Sequence Synthetic primer”
ctatgttcttatctcctcagt

SEQ ID NO: 108 - DNA, 18 bp, Artificial Sequence
  source: /note=“Description of Artificial Sequence Synthetic primer”
ggttccatcctagctctt

SEQ ID NO: 109 - DNA, 18 bp, Artificial Sequence
  source: /note=“Description of Artificial Sequence Synthetic primer”
actcacccactctctgat

SEQ ID NO: 110 - DNA, 19 bp, Artificial Sequence
  source: /note=“Description of Artificial Sequence Synthetic primer”
acccagtgacttacctatg

SEQ ID NO: 111 - DNA, 20 bp, Artificial Sequence
  source: /note=“Description of Artificial Sequence Synthetic primer”
ttcaaatctggaaaggacac

SEQ ID NO: 112 - DNA, 18 bp, Artificial Sequence
  source: /note=“Description of Artificial Sequence Synthetic primer”
cttctggggaagctcatt

SEQ ID NO: 113 - DNA, 19 bp, Artificial Sequence
  source: /note=“Description of Artificial Sequence Synthetic primer”
cagcatctgaaacccatag

SEQ ID NO: 114 - DNA, 19 bp, Artificial Sequence
  source: /note=“Description of Artificial Sequence Synthetic primer”
gtgctctgtgaatgtcatc

SEQ ID NO: 115 - DNA, 20 bp, Artificial Sequence
  source: /note=“Description of Artificial Sequence Synthetic primer”
tacccaggtttccagaatag

SEQ ID NO: 116 - DNA, 19 bp, Artificial Sequence
  source: /note=“Description of Artificial Sequence Synthetic primer”
ctgtagactaccgagctac

SEQ ID NO: 117 - DNA, 16 bp, Artificial Sequence
  source: /note=“Description of Artificial Sequence Synthetic primer”
tcggaggaatgcctga

SEQ ID NO: 118 - DNA, 22 bp, Artificial Sequence
  source: /note=“Description of Artificial Sequence Synthetic primer”
ctaaacagtgggaattctagac

SEQ ID NO: 119 - DNA, 19 bp, Artificial Sequence
  source: /note=“Description of Artificial Sequence Synthetic primer”
ctgctgaagatctggaaga

SEQ ID NO: 120 - DNA, 26 bp, Artificial Sequence
  source: /note=“Description of Artificial Sequence Synthetic primer”
cttcatttatttcattgtataacaca

SEQ ID NO: 121 - DNA, 20 bp, Artificial Sequence
  source: /note=“Description of Artificial Sequence Synthetic primer”
gcattaaaggactgactgaa

SEQ ID NO: 122 - DNA, 18 bp, Artificial Sequence
  source: /note=“Description of Artificial Sequence Synthetic primer”
gatctcactctcaggaga

SEQ ID NO: 123 - DNA, 20 bp, Artificial Sequence
  source: /note=“Description of Artificial Sequence Synthetic primer”
acacctctatcttcaaccaa

SEQ ID NO: 124 - DNA, 19 bp, Artificial Sequence
  source: /note=“Description of Artificial Sequence Synthetic primer”
gctcatctgaaaacaggag

SEQ ID NO: 125 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 126 - protein, 1210 aa, Homo sapiens [sequence omitted]

SEQ ID NO: 127 - protein, 154 aa, Homo sapiens [sequence omitted]

SEQ ID NO: 128 - protein, 308 aa, Homo sapiens
MGEIKVSPDYNWFRGTVPLKKIIVDDDDSKIWSLYDAGPRSIRCPLIFLPPVSGTADVFFRQILALTGWGYRVIALQYPVYWDHLEFCDGFRKLLDHLQLDKVHLFGASLGGFLAQKFAEYTHKSPRVHSLILCNSFSDTSIFNQTWTANSFWLMPAFMLKKIVLGNFSSGPVDPMMADAIDFMVDRLESLGQSELASRLTLNCQNSYVEPHKIRDIPVTIMDVFDQSALSTEAKEEMYKLYPNARRAHLKTGGNFPYLCRSAEVNLYVQIHLLQFHGTKYAAIDPSMVSAEELEVQKGSLGISQEEQ

SEQ ID NO: 129 - protein, 212 aa, Homo sapiens
MNIFRLTGDLSHLAAIVILLLKIWKTRSCAGISGKSQLLFALVFTTRYLDLFTSFISLYNTSMKVIYLACSYATVYLIYLKFKATYDGNHDTFRVEFLVVPVGGLSFLVNHDFSPLEILWTFSIYLESVAILPQLFMISKTGEAETITTHYLFFLGLYRALYLVNWIWRFYFEGFFDLIAVVAGVVQTILYCDFFYLYITKVLKGKKLSLPA

SEQ ID NO: 130 - protein, 4 aa, Unknown
  source: /note=“Description of Unknown ′KDEL′ family peptide motif”
KDEL

SEQ ID NO: 131 - protein, 1408 aa, Homo sapiens; identical to SEQ ID NO: 125
  VARIANT (1058)..(1058): /replace=“Pro”

SEQ ID NO: 132 - DNA, 121 bp, Homo sapiens
gtcccccatcctaactagtggggactctgatatatccagtccattactgcaaaatactgtccacattgacctcagtgctctaaatccagagctggtccaggcagtgcagcatgtagtgatt